(19) United States
(12) Patent Application Publication
(10) Pub. No.: US 2025/0289790 A1
(43) Pub. Date: Sep. 18, 2025

(54) UV LIGHT STABILIZERS

(71) Applicant: BASF SE, Ludwigshafen am Rhein (DE)

(72) Inventors: Barbara WINKLER, Schweizerhalle (Muttenz) (CH); Wolfgang PETER, Ludwigshafen am Rhein (DE); Stephanie BOPP, Schweizerhalle (Muttenz) (CH)

(21) Appl. No.: 18/862,975

(22) PCT Filed: May 2, 2023
(86) PCT No.: PCT/EP2023/061489

§ 371 Date: Nov. 5, 2024

(30) Foreign Application Priority Data
May 6, 2022 (EP) 22171951.1

(51) Int. Cl. C07D 249/20; C07D 251/24; C08K 5/3475; C09D 7/48; C09D 7/63

(52) U.S. Cl. CPC C07D 249/20; C07D 251/24; C08K 5/3475; C09D 7/48; C09D 7/63

(57) ABSTRACT

The presently claimed invention is directed to a product, or a salt thereof obtainable by a process comprising at least the step of: a) reacting at least one compound of formula (I) wherein G* is selected from formula (A), or formula (B) b) with at least one compound of formula (II), and the use of said product as ultraviolet stabilizer, e.g. in a method of protecting a material or coating from light.

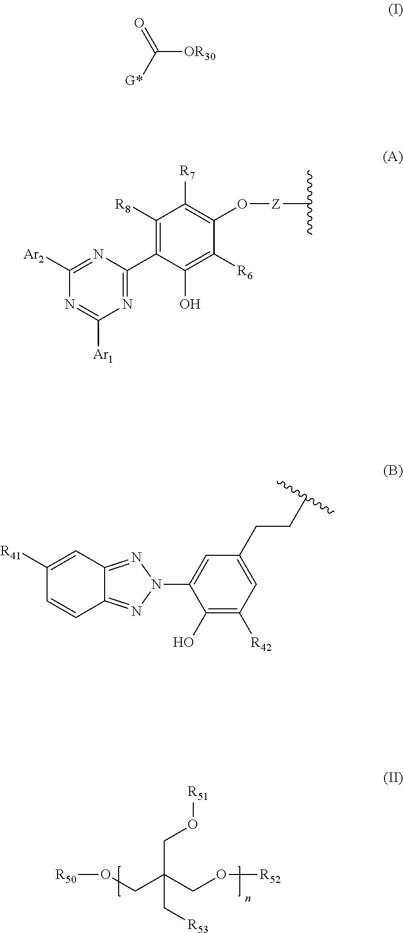

### FIELD OF INVENTION

The presently claimed invention is directed a UV absorbing compounds and process for the preparation thereof.

### BACKGROUND OF THE INVENTION

Benzotriazole derivatives represented by the following formula (C1) belong to a chemical group which are useful as UV absorbers for protecting household products from photolytic and oxidative degradation, as sunscreens in cosmetic compositions, as plastic additives, for protection of coatings from photo-degradation, cracking and delamination, as additives for photographic and printing applications, as additives for electronic applications and recording materials as well as for protecting the ingredients in agriculture applications.

WO 2019006750 A1 describes the use of reactive benzotriazoles based on mono-trimethylolpropane:

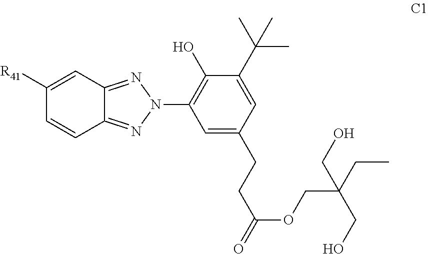

WO 2010130752 A1 describes the following benzotriazole UV absorbers based on trimethylolpropane and pentaerythritol molecule having following structures

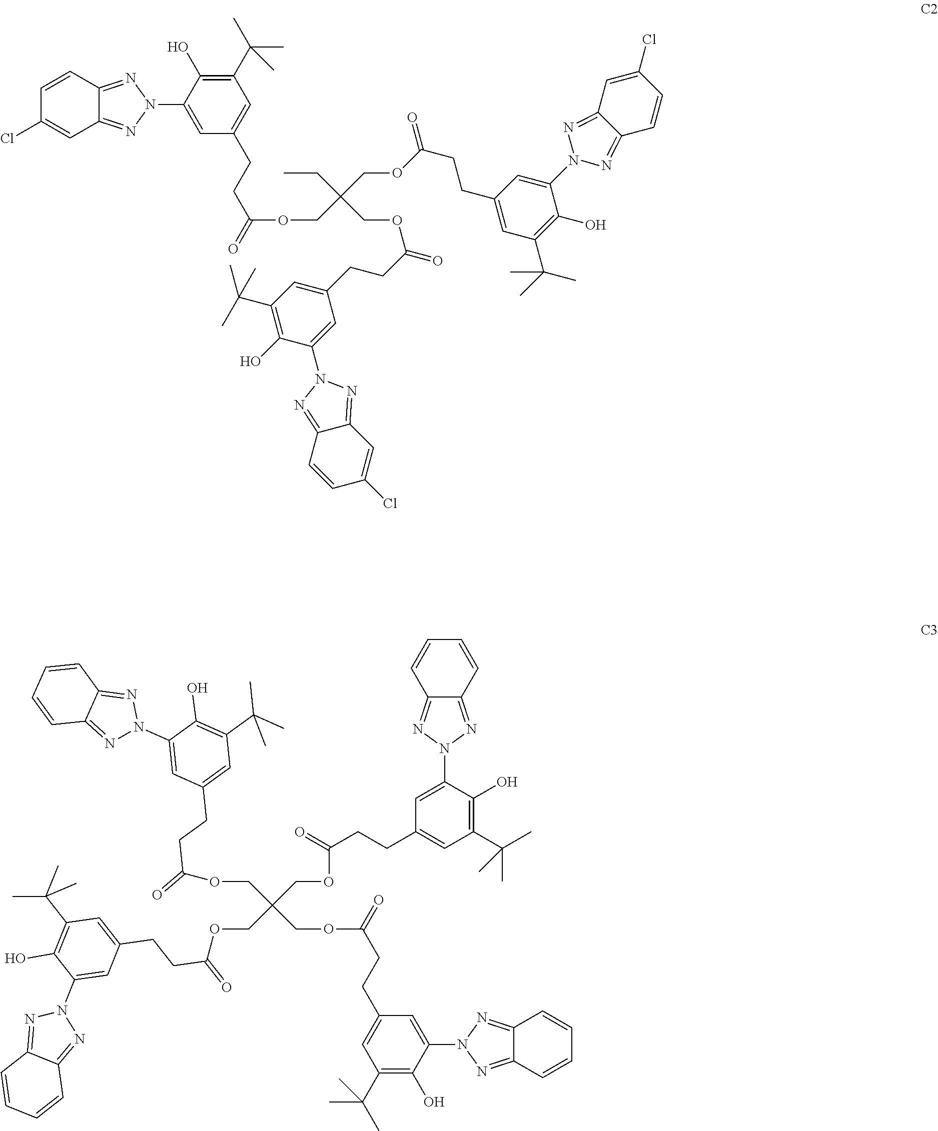 [SMILES: CCC(COC(=O)CCc1cc(-n2nc3ccc(Cl)cc3n2)c(O)c(C(C)(C)C)c1)(COC(=O)CCc1cc(-n2nc3ccc(Cl)cc3n2)c(O)c(C(C)(C)C)c1)COC(=O)CCc1cc(-n2nc3ccc(Cl)cc3n2)c(O)c(C(C)(C)C)c1 ; CC(C)(C)c1cc(CCC(=O)OCC(COC(=O)CCc2cc(-n3nc4ccccc4n3)c(O)c(C(C)(C)C)c2)(COC(=O)CCc2cc(-n3nc4ccccc4n3)c(O)c(C(C)(C)C)c2)COC(=O)CCc2cc(-n3nc4ccccc4n3)c(O)c(C(C)(C)C)c2)cc(-n2nc3ccccc3n2)c1O]

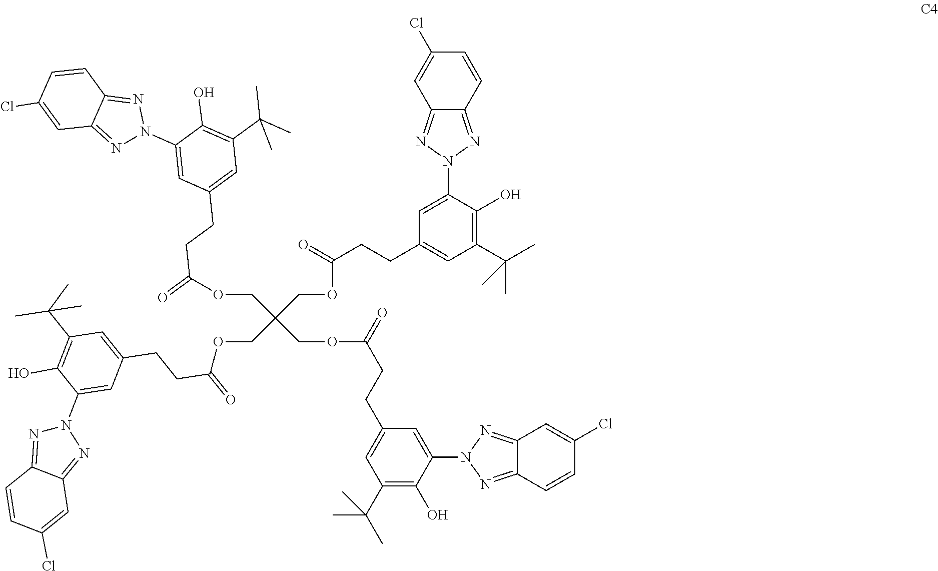 [SMILES: CC(C)(C)c1cc(CCC(=O)OCC(COC(=O)CCc2cc(-n3nc4ccc(Cl)cc4n3)c(O)c(C(C)(C)C)c2)(COC(=O)CCc2cc(-n3nc4ccc(Cl)cc4n3)c(O)c(C(C)(C)C)c2)COC(=O)CCc2cc(-n3nc4ccc(Cl)cc4n3)c(O)c(C(C)(C)C)c2)cc(-n2nc3ccc(Cl)cc3n2)c1O]

However, the problem associated with these products is their low solubility due to which it is difficult to obtain a homogenous mixture in the application product, especially, while using for coating composition. Further, these products have reduced long term stability. Thus, it is desirable to have UV light stabilizers having a solubility of at least 10 g/100 mL xylene and/or liquid/melt at room temperature and/or having long term stability. Therefore, the object is to develop a UV stabilizer product having higher solubilities in organic solvent, which can reduce the usage of environmental hazardous solvents, such as aromatic hydrocarbons. Yet another object of the presently claimed invention is to provide UV stabilizers which are able to provide stability to materials and coating long term compared to known products.

### SUMMARY OF THE INVENTION

Surprisingly it was found that UV absorbers bound to pentaerythritol and trimethylolpropane or similar backbones can improve the solubility of manyfold.

Accordingly, the first aspect of the presently claimed invention is directed to a product, or a salt thereof obtainable by a process comprising at least the step of:
a) reacting at least one compound of formula (I),

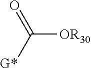

compound of formula (I)
wherein G* is selected from formula (A), or formula (B)

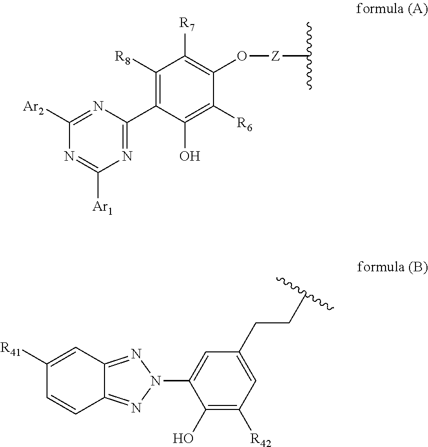

wherein Z is selected from substituted or unsubstituted, linear or branched C₁-C₃₀ alkylene, substituted or unsubstituted, linear or branched 2- to 30-membered hetero alkylene, substituted or unsubstituted, linear or branched C₂-C₂₄ alkenylene, substituted or unsubstituted, linear or branched 3- to 30-membered hetero alkenylene, substituted or unsubstituted C₅-C₂₄ cycloalkylene, or substituted or unsubstituted C₆-C₂₄ arylene,
Ar₁ and Ar₂ are independently of each other a moiety of the formula (C),

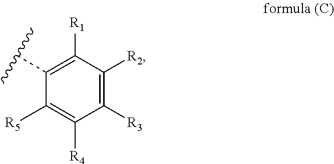

wherein, the dotted line is a single bond between formula (C) and the triazinyl ring of formula (A), and R₁, R₂, R₃, R₄ and R₅ are independently of each other selected from hydrogen, substituted or unsubstituted, linear or branched C₁-C₂₄ alkyl, substituted or unsubstituted C₆-C₂₄ aryl, —OH, —OC₁-C₂₄ alkyl; or a moiety of formula (M),

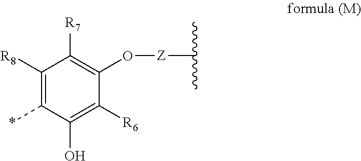

wherein the dotted line is a single bond between formula (M) and the triazinyl ring of formula (A), Z is defined as above, and Z is attached to —C(═O)OR₃₀ and wherein R₃₀ is defined as above,
R₆, R₇ and R₈ are independently of each other selected from hydrogen, or substituted or unsubstituted, linear or branched C₁-C₂₄ alkyl;
R₄₁, and R₄₂ independently of each other, are selected from hydrogen, halogen, substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, or substituted or unsubstituted C₇-C₂₄ arylalkyl,
R₃₀ is selected from hydrogen, substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, substituted or unsubstituted C₇-C₂₄ arylalkyl, or —S(═O)₂R₃₁, wherein R₃₁ is selected from substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₆-C₂₄ aryl, substituted or unsubstituted C₇-C₂₄ arylalkyl,
b) with at least one compound of formula (II)

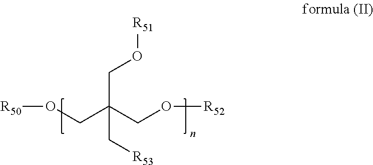

wherein n is an integer from 1 to 20,
wherein R₅₀, R₅₁, and R₅₂ are independently from each other selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, —(C═O)R₅₅, or —[CH₂CHR₅₆O]ₘ—R₅₇;
R₅₃ is selected from hydrogen, substituted or unsubstituted, linear or branched C₁-C₂₄ alkyl, substituted or unsubstituted, linear or branched C₂-C₂₄ alkenyl, or OR₅₉, wherein R₅₉ is selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, —(C═O)R₅₅, or —[CH₂CHR₅₆O]ₘ—R₅₇,
R₅₄ is selected from hydrogen, substituted or unsubstituted, linear or branched C₁-C₂₄ alkyl, substituted or unsubstituted, linear or branched C₂-C₂₄ alkenyl, or —(C═O)—C₁-C₂₄ alkyl;
R₅₅ is selected from substituted or unsubstituted, linear or branched C₁-C₂₄ alkyl, or substituted or unsubstituted, linear or branched C₂-C₂₄ alkenyl;
R₅₆ is selected from hydrogen, or substituted or unsubstituted, linear or branched C₁-C₂₄ alkyl, R₅₇ is selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, or —(C═O)R₅₅;
wherein t is an integer from 1 to 20,
wherein j is an integer from 1 to 20,
wherein m is an integer from 1 to 100,
with the proviso that at least one of R₅₀, R₅₁, R₅₂, R₅₄, R₅₇ and R₅₉ is hydrogen; and if n=1, at least one of R₅₀, R₅₁ and R₅₂ is not hydrogen.

Second aspect of the presently claimed invention is directed to use of the product or a salt thereof according first aspect as ultraviolet stabilizer.

Third aspect of the presently claimed invention is directed a composition comprising a product or a salt thereof according first aspect.

The fourth aspect of the presently claimed invention is directed to a method of protecting a material or coating from light, wherein the method comprises a step of providing the product or salt thereof as UV stabilizer according to first aspect.

### DETAILED DESCRIPTION

Before the present compositions and formulations of the presently claimed invention are described, it is to be understood that this invention is not limited to particular compositions and formulations described, since such compositions and formulation may, of course, vary. It is also to be understood that the terminology used herein is not intended to be limiting, since the scope of the presently claimed invention will be limited only by the appended claims.

If hereinafter a group is defined to comprise at least a certain number of embodiments, this is meant to also encompass a group which preferably consists of these embodiments only. Furthermore, the terms ‘first’, ‘second’, ‘third’ or ‘a’, ‘b’, ‘c’, etc. and the like in the description and in the claims, are used for distinguishing between similar elements and not necessarily for describing a sequential or chronological order. It is to be understood that the terms so used are inter-changeable under appropriate circumstances and that the embodiments of the presently claimed invention described herein are capable of operation in other sequences than described or illustrated herein. In case the terms ‘first’, ‘second’, ‘third’ or ‘(A)’, ‘(B)’ and ‘(C)’ or ‘(a)’, ‘(b)’, ‘(c)’, ‘(d)’, ‘i’, ‘ii’ etc. relate to steps of a method or use or assay there is no time or time interval coherence between the steps, that is, the steps may be carried out simultaneously or there may be time intervals of seconds, minutes, hours, days, weeks, months or even years between such steps, unless otherwise indicated in the application as set forth herein above or below.

Furthermore, the ranges defined throughout the specification include the end values as well i.e. a range of 1 to 10 implies that both 1 and 10 are included in the range. For the avoidance of doubt, applicant shall be entitled to any equivalents according to applicable law.

In the following passages, different aspects of the presently claimed invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to ‘one embodiment’ or ‘an embodiment’ means that a particular feature, structure or characteristic described in connection with the embodiment is included in at least one embodiment of the presently claimed invention. Thus, appearances of the phrases ‘in one embodiment’ or ‘in an embodiment’ in various places throughout this specification are not necessarily all referring to the same embodiment.

Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the presently claimed invention, and form different embodiments, as would be understood by those in the art. For example, in the appended claims, any of the claimed embodiments can be used in any combination.

Accordingly, the presently claimed invention is directed to a product, or a salt thereof obtainable by a process comprising at least the step of:
a) reacting at least one compound of formula (I),

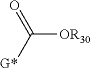

compound of formula (I)
wherein G* is selected from formula (A), or formula (B)

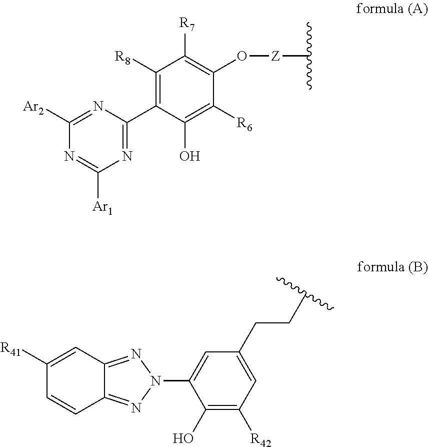

wherein Z is selected from substituted or unsubstituted, linear or branched C₁-C₃₀ alkylene, substituted or unsubstituted, linear or branched 2- to 30-membered hetero alkylene, substituted or unsubstituted, linear or branched C₂-C₂₄ alkenylene, substituted or unsubstituted, linear or branched 3- to 30-membered hetero alkenylene, substituted or unsubstituted C₅-C₂₄ cycloalkylene, or substituted or unsubstituted C₆-C₂₄ arylene,
Ar₁ and Ar₂ are independently of each other a moiety of the formula (C),

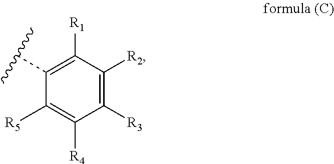

wherein the dotted line is a single bond between formula (C) and the triazinyl ring of formula (A), and R₁, R₂, R₃, R₄ and R₅ are independently of each other selected from hydrogen, substituted or unsubstituted, linear or branched C₁-C₂₄ alkyl, substituted or unsubstituted C₆-C₂₄ aryl, —OH, —OC₁-C₂₄ alkyl; or a moiety of formula (M),

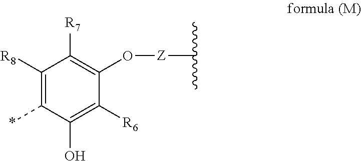

wherein the dotted line is a single bond between formula (M) and the triazinyl ring of formula (A), Z is defined as above, and Z is attached to —C(═O)OR₃₀ and wherein R₃₀ is defined as below,
R₆, R₇ and R₈ are independently of each other selected from hydrogen, or substituted or unsubstituted, linear or branched C₁-C₂₄ alkyl;
R₄₁, and R₄₂ independently of each other, are selected from hydrogen, halogen, substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, or substituted or unsubstituted C₇-C₂₄ arylalkyl,
R₃₀ is selected from hydrogen, substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, substituted or unsubstituted C₇-C₂₄ arylalkyl, or —S(═O)₂R₃₁, wherein R₃₁ is selected from substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₆-C₂₄ aryl, substituted or unsubstituted C₇-C₂₄ arylalkyl,
b) with at least one compound of formula (II)

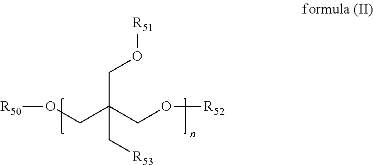

wherein n is an integer from 1 to 20,
wherein R₅₀, R₅₁, and R₅₂ are independently from each other selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, —(C═O)R₅₅, or —[CH₂CHR₅₆O]ₘ—R₅₇;
R₅₃ is selected from hydrogen, substituted or unsubstituted, linear or branched C₁-C₂₄ alkyl, substituted or unsubstituted, linear or branched C₂-C₂₄ alkenyl, or OR₅₉,
wherein R₅₉ is selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, —(C═O)R₅₅, or —[CH₂CHR₅₆O]ₘ—R₅₇,
R₅₄ is selected from hydrogen, substituted or unsubstituted, linear or branched C₁-C₂₄ alkyl, substituted or unsubstituted, linear or branched C₂-C₂₄ alkenyl, or —(C═O)—C₁-C₂₄ alkyl;
R₅₅ is selected from substituted or unsubstituted, linear or branched C₁-C₂₄ alkyl, or substituted or unsubstituted, linear or branched C₂-C₂₄ alkenyl;
R₅₆ is selected from hydrogen, or substituted or unsubstituted, linear or branched C₁-C₂₄ alkyl,
R₅₇ is selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, or —(C═O)R₅₅;
wherein t is an integer from 1 to 20,
wherein j is an integer from 1 to 20,
wherein m is an integer from 1 to 100,
with the proviso that at least one of R₅₀, R₅₁, R₅₂, R₅₄, R₅₇ and R₅₉ is hydrogen; and if n=1, at least one of R₅₀, R₅₁ and R₅₂ is not hydrogen.

Preferably, the presently claimed invention is directed to a product, or a salt thereof obtainable by a process comprising at least the step of:
a) reacting at least one compound of formula (I),

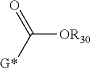

compound of formula (I)
wherein G* is selected from formula (A), or formula (B)

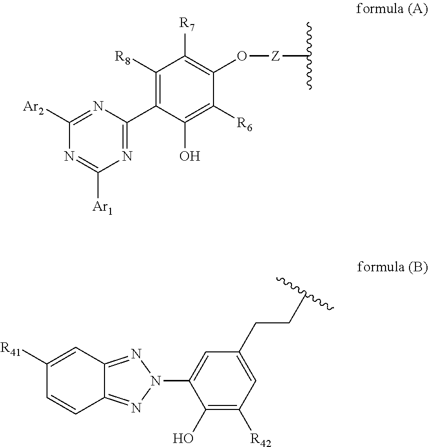

wherein Z is selected from substituted or unsubstituted, linear or branched C₁-C₃₀ alkylene, substituted or unsubstituted, linear or branched 2- to 30-membered hetero alkylene, substituted or unsubstituted, linear or branched C₂-C₂₄ alkenylene,
Ar₁ and Ar₂ are independently of each other a moiety of the formula (C),

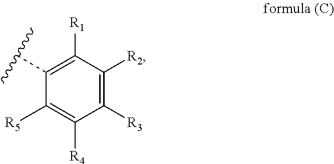

wherein the dotted line is a single bond between formula (C) and the triazinyl ring of formula (A), and R₁, R₂, R₃, R₄ and R₅ are independently of each other selected from hydrogen, substituted or unsubstituted, linear or branched C₁-C₂₄ alkyl, substituted or unsubstituted C₆-C₂₄ aryl, —OH, —OC₁-C₂₄ alkyl; or a moiety of formula (M),

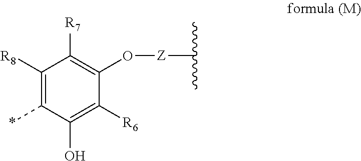

wherein the dotted line is a single bond between formula (M) and the triazinyl ring of formula (A), Z is defined as above, and Z is attached to —C(═O)OR₃₀ and wherein R₃₀ is defined as above,
R₆, R₇ and R₈ are independently of each other selected from hydrogen, or substituted or unsubstituted, linear or branched C₁-C₂₄ alkyl;
R₄₁, and R₄₂ independently of each other, are selected from hydrogen, halogen, substituted or unsubstituted, linear or branched C₁-C₂₄ alkyl, substituted or unsubstituted, linear or branched C₂-C₂₄ alkenyl, substituted or unsubstituted C₆-C₂₄ aryl, or substituted or unsubstituted C₇-C₂₄ arylalkyl,
R₃₀ is selected from hydrogen, substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, substituted or unsubstituted C₇-C₂₄ arylalkyl, or —S(═O)₂R₃₁, wherein R₃₁ is selected from substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₆-C₂₄ aryl, substituted or unsubstituted C₇-C₂₄ arylalkyl,
b) with at least one compound of formula (II)

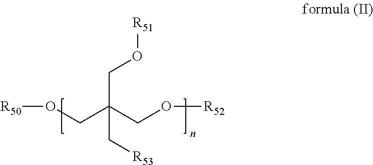

wherein n is an integer from 1 to 20,
wherein R₅₀, R₅₁, and R₅₂ are independently from each other selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, —(C═O)R₅₅, or —[CH₂CHR₅₆O]ₘ—R₅₇;
R₅₃ is selected from hydrogen, substituted or unsubstituted, linear or branched C₁-C₂₄ alkyl, substituted or unsubstituted, linear or branched C₂-C₂₄ alkenyl, or OR₅₉,
wherein R₅₉ is selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, —(C═O)R₅₅, or —[CH₂CHR₅₆O]ₘ—R₅₇,
R₅₄ is selected from hydrogen, substituted or unsubstituted, linear or branched C₁-C₂₄ alkyl, substituted or unsubstituted, linear or branched C₂-C₂₄ alkenyl, or —(C═O)—C₁-C₂₄ alkyl;
R₅₅ is selected from substituted or unsubstituted, linear or branched C₁-C₂₄ alkyl, or substituted or unsubstituted, linear or branched C₂-C₂₄ alkenyl;
R₅₆ is selected from hydrogen, or substituted or unsubstituted, linear or branched C₁-C₂₄ alkyl,
R₅₇ is selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, or —(C═O)R₅₅;
wherein t is an integer from 1 to 20,
wherein j is an integer from 1 to 20,
wherein m is an integer from 1 to 100,
with the proviso that at least one of R₅₀, R₅₁, R₅₂, R₅₄, R₅₇ and R₅₉ is hydrogen; and if n=1, at least one of R₅₀, R₅₁ and R₅₂ is not hydrogen.

More preferably, the presently claimed invention is directed to a product, or a salt thereof obtainable by a process comprising at least the step of:
a) reacting at least one compound of formula (I),

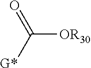

compound of formula (I)
wherein G* is selected from formula (A), or formula (B)

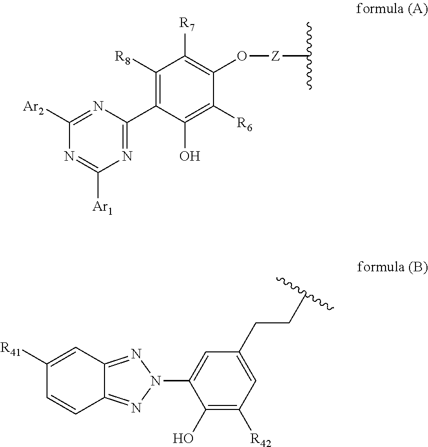

wherein Z is selected from substituted or unsubstituted, linear or branched C₁-C₃₀ alkylene, substituted or unsubstituted, linear or branched 2- to 30-membered hetero alkylene, substituted or unsubstituted, linear or branched C₂-C₂₄ alkenylene,
Ar₁ and Ar₂ are independently of each other a moiety of the formula (C),

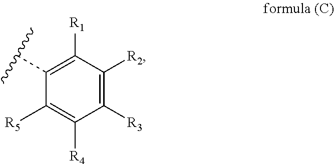

wherein the dotted line is a single bond between formula (C) and the triazinyl ring of formula (A), and R₁, R₂, R₃, R₄ and R₅ are independently of each other selected from hydrogen, substituted or unsubstituted, linear or branched C₁-C₂₄ alkyl, —OH, —OC₁-C₂₄ alkyl; or a moiety of formula (M),

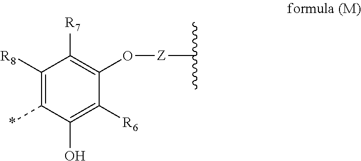

wherein the dotted line is a single bond between formula (M) and the triazinyl ring of formula (A), Z is defined as above, and Z is attached to —C(═O)OR₃₀ and wherein R₃₀ is defined as below,
R₆, R₇ and R₈ are independently of each other selected from hydrogen, or substituted or unsubstituted, linear or branched C₁-C₂₄ alkyl;
R₄₁, and R₄₂ independently of each other, are selected from hydrogen, halogen, substituted or unsubstituted, linear or branched C₁-C₂₄ alkyl, substituted or unsubstituted, linear or branched C₂-C₂₄ alkenyl,
R₃₀ is selected from hydrogen, substituted or unsubstituted, linear or branched C₁-C₂₄ alkyl, substituted or unsubstituted, linear or branched C₂-C₂₄ alkenyl, substituted or unsubstituted C₆-C₂₄ aryl, substituted or unsubstituted C₇-C₂₄ arylalkyl, or —S(═O)₂R₃₁, wherein R₃₁ is selected from substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₆-C₂₄ aryl, substituted or unsubstituted C₇-C₂₄ arylalkyl,
b) with at least one compound of formula (II)

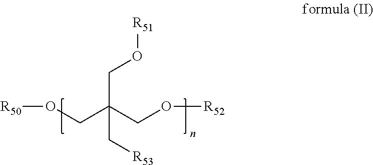

wherein n is an integer from 1 to 10,
wherein R₅₀, R₅₁, and R₅₂ are independently from each other selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, —(C═O)R₅₅, or —[CH₂CHR₅₆O]ₘ—R₅₇;
R₅₃ is selected from hydrogen, substituted or unsubstituted, linear or branched C₁-C₂₄ alkyl, substituted or unsubstituted, linear or branched C₂-C₂₄ alkenyl, or OR₅₉,
wherein R₅₉ is selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]l-R₅₄, —(C═O)R₅₅, or —[CH₂CHR₅₆O]ₘ—R₅₇,
R₅₄ is selected from hydrogen, substituted or unsubstituted, linear or branched C₁-C₂₄ alkyl, substituted or unsubstituted, linear or branched C₂-C₂₄ alkenyl, or —(C═O)—C₁-C₂₄ alkyl;
R₅₅ is selected from substituted or unsubstituted, linear or branched C₁-C₂₄ alkyl, or substituted or unsubstituted, linear or branched C₂-C₂₄ alkenyl;
R₅₆ is selected from hydrogen, or substituted or unsubstituted, linear or branched C₁-C₂₄ alkyl,
R₅₇ is selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, or —(C═O)R₅₅;
wherein t is an integer from 1 to 20,
wherein j is an integer from 1 to 20,
wherein m is an integer from 1 to 100,
with the proviso that at least one of R₅₀, R₅₁, R₅₂, R₅₄, R₅₇ and R₅₉ is hydrogen; and if n=1, at least one of R₅₀, R₅₁ and R₅₂ is not hydrogen.

Even more preferably, the presently claimed invention is directed to a product, or a salt thereof obtainable by a process comprising at least the step of:
a) reacting at least one compound of formula (I),

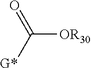

compound of formula (I)
wherein G* is selected from formula (A), or formula (B)

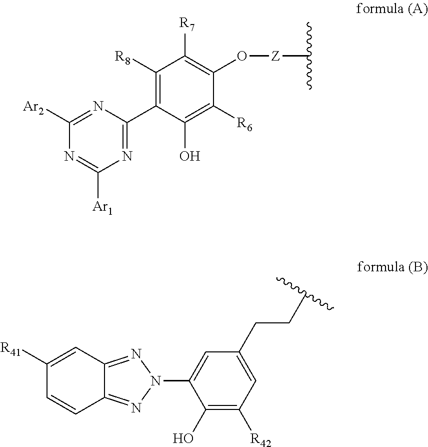

wherein Z is selected from substituted or unsubstituted, linear or branched C₁-C₃₀ alkylene, substituted or unsubstituted, linear or branched 2- to 30-membered hetero alkylene, substituted or unsubstituted, linear or branched C₂-C₂₄ alkenylene,
Ar₁ and Ar₂ are independently of each other a moiety of the formula (C),

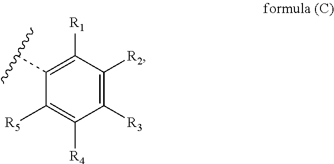

wherein the dotted line is a single bond between formula (C) and the triazinyl ring of formula (A), and R₁, R₂, R₃, R₄ and R₅ are independently of each other selected from hydrogen, substituted or unsubstituted, linear or branched C₁-C₂₄ alkyl, —OH, —OC₁-C₂₄ alkyl; or a moiety of formula (M),

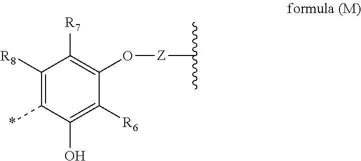

wherein the dotted line is a single bond between formula (M) and the triazinyl ring of formula (A), Z is defined as above, and Z is attached to —C(═O)OR₃₀ and wherein R₃₀ is defined as below,
R₆, R₇ and R₈ are independently of each other selected from hydrogen, or substituted or unsubstituted, linear or branched C₁-C₂₄ alkyl;
R₄₁, and R₄₂ independently of each other, are selected from hydrogen, halogen, substituted or unsubstituted, linear or branched C₁-C₂₄ alkyl, substituted or unsubstituted, linear or branched C₂-C₂₄ alkenyl,
R₃₀ is selected from hydrogen, substituted or unsubstituted, linear or branched C₁-C₂₄ alkyl, substituted or unsubstituted, linear or branched C₂-C₂₄ alkenyl, substituted or unsubstituted C₆-C₂₄ aryl, or substituted or unsubstituted C₇-C₂₄ arylalkyl,
b) with at least one compound of formula (II)

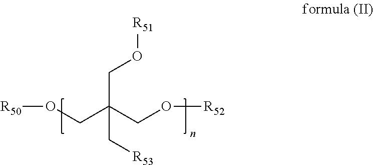

wherein n is an integer from 1 to 8,
wherein R₅₀, R₅₁, and R₅₂ are independently from each other selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, —(C═O)R₅₅, or —[CH₂CHR₅₆O]ₘ—R₅₇;
R₅₃ is selected from hydrogen, substituted or unsubstituted, linear or branched C₁-C₂₄ alkyl, substituted or unsubstituted, linear or branched C₂-C₂₄ alkenyl, or OR₅₉,
wherein R₅₉ is selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, —(C═O)R₅₅, or —[CH₂CHR₅₆O]ₘ—R₅₇,
R₅₄ is selected from hydrogen, substituted or unsubstituted, linear or branched C₁-C₂₄ alkyl, substituted or unsubstituted, linear or branched C₂-C₂₄ alkenyl, or —(C═O)—C₁-C₂₄ alkyl;
R₅₅ is selected from substituted or unsubstituted, linear or branched C₁-C₂₄ alkyl, or substituted or unsubstituted, linear or branched C₂-C₂₄ alkenyl;
R₅₆ is selected from hydrogen, or substituted or unsubstituted, linear or branched C₁-C₂₄ alkyl,
R₅₇ is selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, or —(C═O)R₅₅;
wherein t is an integer from 1 to 20,
wherein j is an integer from 1 to 20,
wherein m is an integer from 1 to 100,
with the proviso that at least one of R₅₀, R₅₁, R₅₂, R₅₄, R₅₇ and R₅₉ is hydrogen; and if n=1, at least one of R₅₀, R₅₁ and R₅₂ is not hydrogen.

Most preferably, the presently claimed invention is directed to a product, or a salt thereof obtainable by a process comprising at least the step of:
a) reacting at least one compound of formula (I),

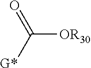

compound of formula (I)
wherein G* is formula (B)

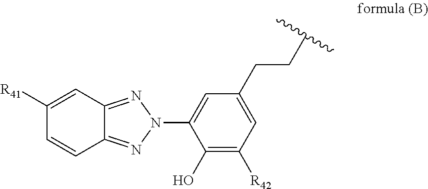

R₄₁, and R₄₂ independently of each other, are selected from hydrogen, halogen, substituted or unsubstituted, linear or branched C₁-C₂₄ alkyl, substituted or unsubstituted, linear or branched C₂-C₂₄ alkenyl, or substituted or unsubstituted C₇-C₂₄ arylalkyl.
R₃₀ is selected from hydrogen, substituted or unsubstituted, linear or branched C₁-C₂₄ alkyl, substituted or unsubstituted, linear or branched C₂-C₂₄ alkenyl, substituted or unsubstituted C₆-C₂₄ aryl, or substituted or unsubstituted C₇-C₂₄ arylalkyl,
b) with at least one compound of formula (II)

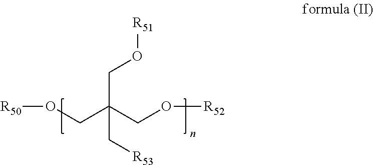

wherein n is an integer from 1 to 5,
wherein R₅₀, R₅₁, and R₅₂ are independently from each other selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, —(C═O)R₅₅, or —[CH₂CHR₅₆O]ₘ—R₅₇;
R₅₃ is selected from hydrogen, substituted or unsubstituted, linear or branched C₁-C₂₄ alkyl, substituted or unsubstituted, linear or branched C₂-C₂₄ alkenyl, or OR₅₉,
wherein R₅₉ is selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, —(C═O)R₅₅, or —[CH₂CHR₅₆O]ₘ—R₅₇,
R₅₄ is selected from hydrogen, substituted or unsubstituted, linear or branched C₁-C₂₄ alkyl, substituted or unsubstituted, linear or branched C₂-C₂₄ alkenyl, or —(C═O)—C₁-C₂₄ alkyl;
R₅₅ is selected from substituted or unsubstituted, linear or branched C₁-C₂₄ alkyl, or substituted or unsubstituted, linear or branched C₂-C₂₄ alkenyl;
R₅₆ is selected from hydrogen, or substituted or unsubstituted, linear or branched C₁-C₂₄ alkyl,
R₅₇ is selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, or —(C═O)R₅₅;
wherein t is an integer from 1 to 20,
wherein j is an integer from 1 to 20,
wherein m is an integer from 1 to 100,
with the proviso that at least one of R₅₀, R₅₁, R₅₂, R₅₄, R₅₇ and R₅₉ is hydrogen; and if n=1, at least one of R₅₀, R₅₁ and R₅₂ is not hydrogen.

In particular, the presently claimed invention is directed to a product, or a salt thereof obtainable by a process comprising at least the step of:
a) reacting at least one compound of formula (I),

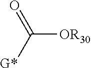

compound of formula (I)
wherein G* is formula (B)

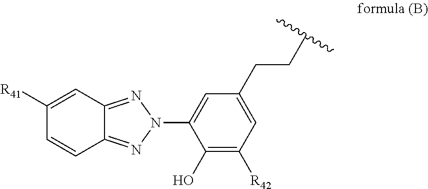

R₄₁, and R₄₂ independently of each other, are selected from hydrogen, or substituted or unsubstituted, linear or branched C₁-C₂₄ alkyl,
R₃₀ is selected from hydrogen, or substituted or unsubstituted, linear or branched C₁-C₂₄ alkyl, b) with at least one compound of formula (II)

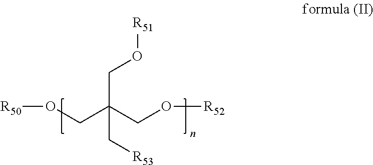

wherein n is an integer from 2 to 5,
wherein R₅₀, R₅₁, and R₅₂ are independently from each other selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, —(C═O)R₅₅, or —[CH₂CHR₅₆O]ₘ—R₅₇;
R₅₃ is selected from hydrogen, substituted or unsubstituted, linear or branched C₁-C₂₄ alkyl, substituted or unsubstituted, linear or branched C₂-C₂₄ alkenyl, or OR₅₉,
wherein R₅₉ is selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, —(C═O)R₅₅, or —[CH₂CHR₅₆O]ₘ—R₅₇,
R₅₄ is selected from hydrogen, substituted or unsubstituted, linear or branched C₁-C₂₄ alkyl, substituted or unsubstituted, linear or branched C₂-C₂₄ alkenyl, or —(C═O)—C₁-C₂₄ alkyl;
R₅₅ is selected from substituted or unsubstituted, linear or branched C₁-C₂₄ alkyl, or substituted or unsubstituted, linear or branched C₂-C₂₄ alkenyl;
R₅₆ is selected from hydrogen, or substituted or unsubstituted, linear or branched C₁-C₂₄ alkyl,
R₅₇ is selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, or —(C═O)R₅₅;
wherein t is an integer from 1 to 20,
wherein j is an integer from 1 to 20,
wherein m is an integer from 1 to 100,
with the proviso that at least one of R₅₀, R₅₁, R₅₂, R₅₄, R₅₇ and R₅₉ is hydrogen; and if n=1, at least one of R₅₀, R₅₁ and R₅₂ is not hydrogen.

In another preferred embodiment, according to the presently claimed invention,
wherein G* is

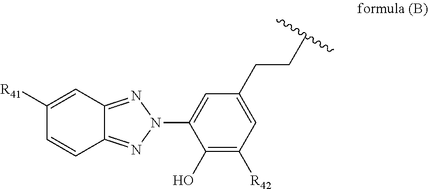

R₄₁, and R₄₂ independently of each other, are selected from hydrogen, halogen, substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, or substituted or unsubstituted C₇-C₂₄ arylalkyl.

In another preferred embodiment, according to the presently claimed invention, the R₄₁, and R₄₂ independently of each other, are selected from hydrogen, halogen, substituted or unsubstituted, linear or branched C₁-C₂₄ alkyl, or substituted or unsubstituted, linear or branched C₂-C₂₄ alkenyl or substituted or unsubstituted C₇-C₂₄ arylalkyl.

In another preferred embodiment, according to the presently claimed invention, at least one compound of formula (II) is

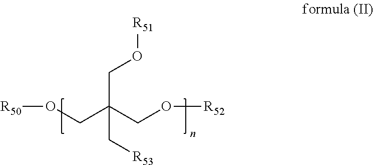

wherein n is an integer from 1 to 10;
wherein R₅₀, R₅₁, and R₅₂ are independently from each other selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, —(C═O)R₅₅, or —[CH₂CHR₅₆O]ₘ—R₅₇;
R₅₃ is selected from hydrogen, substituted or unsubstituted, linear or branched C₁-C₂₄ alkyl, substituted or unsubstituted, linear or branched C₂-C₂₄ alkenyl, or OR₅₉,
wherein R₅₉ is selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, —(C═O)R₅₅, or —[CH₂CHR₅₆O]ₘ—R₅₇,
R₅₄ is selected from hydrogen, substituted or unsubstituted, linear or branched C₁-C₂₄ alkyl, or —(C═O)—C₁₋₂₄ alkyl;
R₅₅ is substituted or unsubstituted, linear or branched C₁-C₂₄ alkyl;
R₅₆ is selected from H, or substituted or unsubstituted, linear or branched C₁-C₂₄ alkyl,
R₅₇ is selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, or —(C═O)R₅₅;
wherein t is an integer from 1 to 10,
wherein j is an integer from 1 to 10,
wherein m is an integer from 1 to 50,
with the proviso that at least one of R₅₀, R₅₁, R₅₂, R₅₄ and R₅₇ is hydrogen; and if n=1, at least one of R₅₀, R₅₁ and R₅₂ is not hydrogen.

In another preferred embodiment, according to the presently claimed invention, wherein n is an integer from 2 to 5;
wherein R₅₀, R₅₁, and R₅₂ are independently from each other selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, —(C═O)R₅₅, or —[CH₂CHR₅₆O]ₘ—R₅₇;
R₅₃ is selected from hydrogen, substituted or unsubstituted, linear or branched C₁₋₆ alkyl, substituted or unsubstituted, linear or branched C₂₋₆ alkenyl, or OR₅₉,
wherein R₅₉ is selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, —(C═O)R₅₅, or —[CH₂CHR₅₆O]ₘ—R₅₇,
R₅₄ is selected from hydrogen, or substituted or unsubstituted, linear or branched C₁-C₂₄ alkyl;
R₅₅ is substituted or unsubstituted, linear or branched C₁-C₂₄ alkyl;
R₅₆ is selected from H, or substituted or unsubstituted, linear or branched C₁₋₆ alkyl,
R₅₇ is selected from hydrogen, substituted or unsubstituted, linear or branched C₁-C₂₄, —[(C═O)—(CH₂)ₜ—O]ⱼ—R₅₄, or —(C═O)R₅₅;
wherein t is an integer from 1 to 5,
wherein j is an integer from 1 to 5,
wherein m is an integer from 1 to 20, with the proviso that at least one of R₅₀, R₅₁, R₅₂, R₅₄ and R₅₇ is hydrogen; and if n=1, at least one of R₅₀, R₅₁ and R₅₂ is not hydrogen.

In another preferred embodiment, according to the presently claimed invention, wherein n is an integer from 2 to 3;
wherein R₅₀, R₅₁, and R₅₂ are independently from each other selected from hydrogen, substituted or unsubstituted, linear or branched C₁-C₂₄ alkyl, or —[(C═O)—(CH₂)ₜ—O]ⱼ—R₅₄;
R₅₃ is selected from hydrogen, C₁ alkyl, or OR₅₉, wherein R₅₉ is as defined as above,
R₅₄ is selected from hydrogen, or substituted or unsubstituted, linear or branched C₁₋₆ alkyl;
R₅₅ is substituted or unsubstituted, linear or branched C₁₋₆ alkyl;
R₅₆ is selected from H, or C₁ alkyl,
R₅₇ is selected from hydrogen, substituted or unsubstituted, linear or branched C₁-C₂₄ alkyl, or —[(C═O)—(CH₂)ₜ—O]ⱼ—R₅₄,
wherein t is an integer from 3 to 5,
wherein j is an integer from 1 to 2,
wherein m is an integer from 4 to 16,
with the proviso that at least one of R₅₀, R₅₁, R₅₂, R₅₄ and R₅₇ is hydrogen; and if n=1, at least one of R₅₀, R₅₁ and R₅₂ is not hydrogen.

In another preferred embodiment, the compound of formula (I) according to presently claimed invention is selected from methyl 3-[3-(benzotriazol-2-yl)-5-tert-butyl-4-hydroxy-phenyl]propanoate, ethyl 3-[3-(benzotriazol-2-yl)-5-tert-butyl-4-hydroxy-phenyl]propanoate, 3-[3-(benzotriazol-2-yl)-5-tert-butyl-4-hydroxy-phenyl]propanoic acid, 3-[3-tert-butyl-5-(5-chlorobenzotriazol-2-yl)-4-hydroxy-phenyl]propanoic acid, methyl 3-[3-tert-butyl-5-(5-chlorobenzotriazol-2-yl)-4-hydroxy-phenyl]propanoate, ethyl 3-[3-tert-butyl-5-(5-chlorobenzotriazol-2-yl)-4-hydroxy-phenyl]propanoate, methyl 2-[4-[4,6-bis(2,4-dimethylphenyl)-1,3,5-triazin-2-yl]-3-hydroxy-phenoxy]propanoate, methyl 2-[4-[4,6-bis(4-phenylphenyl)-1,3,5-triazin-2-yl]-3-hydroxy-phenoxy]propanoate, methyl 2-[4-(4,6-diphenyl-1,3,5-triazin-2-yl)-3-hydroxy-phenoxy]propanoate, ethyl 2-[4-[4,6-bis(2,4-di-methylphenyl)-1,3,5-triazin-2-yl]-3-hydroxy-phenoxy]-3-butoxy-propanoate, ethyl 2-[4-(4,6-di-phenyl-1,3,5-triazin-2-yl)-3-hydroxy-phenoxy]octanoate, methyl 2-[4-(4,6-diphenyl-1,3,5-triazin-2-yl)-3-hydroxy-phenoxy]butanoate, methyl 2-[4-[4,6-bis(2,4-dimethylphenyl)-1,3,5-triazin-2-yl]-3-hydroxy-phenoxy]-2-methyl-propanoate, methyl 2-[4-[4,6-bis(2,4-dimethylphenyl)-1,3,5-triazin-2-yl]-3-hydroxy-phenoxy]octanoate, methyl 2-[4-[4,6-bis(2,4-dimethylphenyl)-1,3,5-triazin-2-yl]-3-hydroxy-phenoxy]hexanoate, methyl 2-[4-[4,6-bis(2,4-dihydroxyphenyl)-1,3,5-triazin-2-yl]-3-hydroxy-phenoxy]propanoate, isooctyl 2-[4-[4,6-bis[2-hydroxy-4-(1-methyl-2-octoxy-2-oxo-ethoxy)phenyl]-1,3,5-triazin-2-yl]-3-hydroxy-phenoxy]propanoate (tinuvin 477), isooctyl 2-[4-[4,6-bis(4-phenylphenyl)-1,3,5-triazin-2-yl]-3-hydroxy-phenoxy]propanoate (tinuvin 479), octyl 2-[4-[4,6-bis[2-hydroxy-4-(1-methyl-2-octoxy-2-oxo-ethoxy)phenyl]-1,3,5-triazin-2-yl]-3-hydroxy-phenoxy]propanoate (tinuvin 477), 6-methylheptyl 2-[4-[4,6-bis(4-phenylphenyl)-1,3,5-triazin-2-yl]-3-hydroxy-phenoxy]propanoate (tinuvin 479), octyl 2-[4-[4,6-bis(4-phenylphenyl)-1,3,5-triazin-2-yl]-3-hydroxy-phenoxy]propanoate, methyl 2-[2-[4-[4,6-bis(2,4-dimethylphenyl)-1,3,5-triazin-2-yl]-3-hydroxy-phenoxy]ethoxy]propanoate, methyl 2-[4-[4,6-bis(4-phenylphenyl)-1,3,5-triazin-2-yl]-3-hydroxy-phenoxy]-3-butoxy-propanoate, methyl 2-[3-hydroxy-4-[4-[2-hydroxy-4-(1-methoxycarbonylpropoxy)phenyl]-6-(4-methoxyphenyl)-1,3,5-triazin-2-yl]phenoxy]butanoate, methyl 2-[4-[4,6-bis(4-butoxy-2-hydroxy-phenyl)-1,3,5-triazin-2-yl]-3-hydroxy-phenoxy]propanoate, methyl 2-[4-[4,6-bis(4-hexoxy-2-hydroxy-3-methyl-phenyl)-1,3,5-triazin-2-yl]-3-hydroxy-2-methyl-phenoxy]propanoate, ethyl 2-[4-[4,6-bis(4-phenylphenyl)-1,3,5-triazin-2-yl]-3-hydroxy-phenoxy]butanoate, methyl 2-[4-[4,6-bis(4-phenylphenyl)-1,3,5-triazin-2-yl]-3-hydroxy-phenoxy]octanoate, methyl 2-[4-[4,6-bis[2-hydroxy-4-(1-methoxycarbonylpropoxy)phenyl]-1,3,5-triazin-2-yl]-3-hydroxy-phenoxy]butanoate, methyl 2-[4-[4,6-bis(2,4-dimethylphenyl)-1,3,5-triazin-2-yl]-3-hydroxy-phenoxy]butanoate, ethyl 2-[4-[4,6-bis(4-phenylphenyl)-1,3,5-triazin-2-yl]-3-hydroxy-phenoxy]propanoate, methyl 2-[4-[4,6-bis[2-hydroxy-4-(2-methoxy-1-methyl-2-oxo-ethoxy)phenyl]-1,3,5-triazin-2-yl]-3-hydroxy-phenoxy]propanoate, methyl 2-[3-hydroxy-4-[4-[2-hydroxy-4-(2-methoxy-1-methyl-2-oxo-ethoxy)phenyl]-6-(4-methoxyphenyl)-1,3,5-triazin-2-yl]phenoxy]propanoate, or a combination of two or more thereof, more preferably the compound of formula (I) according to presently claimed invention is selected from methyl 3-[3-(benzotriazol-2-yl)-5-tert-butyl-4-hydroxy-phenyl]propanoate, ethyl 3-[3-(benzotriazol-2-yl)-5-tert-butyl-4-hydroxy-phenyl]propanoate, 3-[3-(benzotriazol-2-yl)-5-tert-butyl-4-hydroxy-phenyl]propanoic acid, 3-[3-tert-butyl-5-(5-chlorobenzotriazol-2-yl)-4-hydroxy-phenyl]propanoic acid, methyl 3-[3-tert-butyl-5-(5-chlorobenzotriazol-2-yl)-4-hydroxy-phenyl]propanoate, ethyl 3-[3-tert-butyl-5-(5-chlorobenzotriazol-2-yl)-4-hydroxy-phenyl]propanoate, methyl 2-[4-[4,6-bis(2,4-dimethylphenyl)-1,3,5-triazin-2-yl]-3-hydroxy-phenoxy]propanoate, methyl 2-[4-[4,6-bis(4-phenylphenyl)-1,3,5-triazin-2-yl]-3-hydroxy-phenoxy]propanoate, methyl 2-[4-(4,6-diphenyl-1,3,5-triazin-2-yl)-3-hydroxy-phenoxy]propanoate, methyl 2-[4-(4,6-diphenyl-1,3,5-triazin-2-yl)-3-hydroxy-phenoxy]butanoate, methyl 2-[4-[4,6-bis(2,4-dimethylphenyl)-1,3,5-triazin-2-yl]-3-hydroxy-phenoxy]-2-methyl-propanoate, methyl 2-[4-[4,6-bis(2,4-dimethylphenyl)-1,3,5-triazin-2-yl]-3-hydroxy-phenoxy]octanoate, methyl 2-[4-[4,6-bis(2,4-dimethylphenyl)-1,3,5-triazin-2-yl]-3-hydroxy-phenoxy]hexanoate, methyl 2-[4-[4,6-bis(2,4-dihydroxyphenyl)-1,3,5-triazin-2-yl]-3-hydroxy-phenoxy]propanoate, isooctyl 2-[4-[4,6-bis[2-hydroxy-4-(1-methyl-2-octoxy-2-oxo-ethoxy)phenyl]-1,3,5-triazin-2-yl]-3-hydroxy-phenoxy]propanoate (tinuvin 477), isooctyl 2-[4-[4,6-bis(4-phenylphenyl)-1,3,5-triazin-2-yl]-3-hydroxy-phenoxy]propanoate (tinuvin 479), methyl 2-[2-[4-[4,6-bis(2,4-dimethylphenyl)-1,3,5-triazin-2-yl]-3-hydroxy-phenoxy]ethoxy]propanoate, methyl 2-[4-[4,6-bis(4-phenylphenyl)-1,3,5-triazin-2-yl]-3-hydroxy-phenoxy]-3-butoxy-propanoate, methyl 2-[3-hydroxy-4-[4-[2-hydroxy-4-(1-methoxycarbonylpropoxy)phenyl]-6-(4-methoxyphenyl)-1,3,5-triazin-2-yl]phenoxy]butanoate, methyl 2-[4-[4,6-bis(4-butoxy-2-hydroxy-phenyl)-1,3,5-triazin-2-yl]-3-hydroxy-phenoxy]propanoate, methyl 2-[4-[4,6-bis(4-hexoxy-2-hydroxy-3-methyl-phenyl)-1,3,5-triazin-2-yl]-3-hydroxy-2-methyl-phenoxy]propanoate, ethyl 2-[4-[4,6-bis(4-phenylphenyl)-1,3,5-triazin-2-yl]-3-hydroxy-phenoxy]butanoate, methyl 2-[4-[4,6-bis(4-phenylphenyl)-1,3,5-triazin-2-yl]-3-hydroxy-phenoxy]octanoate, methyl 2-[4-[4,6-bis[2-hydroxy-4-(1-methoxycarbonylpropoxy)phenyl]-1,3,5-triazin-2-yl]-3-hydroxy-phenoxy]butanoate, methyl 2-[4-[4,6-bis(2,4-dimethylphenyl)-1,3,5-triazin-2-yl]-3-hydroxy-phenoxy]butanoate, ethyl 2-[4-[4,6-bis(4-phenylphenyl)-1,3,5-triazin-2-yl]-3-hydroxy-phenoxy]propanoate, methyl 2-[4-[4,6-bis[2-hydroxy-4-(2-methoxy-1-methyl-2-oxo-ethoxy)phenyl]-1,3,5-triazin-2-yl]-3-hydroxy-phenoxy]propanoate, methyl 2-[3-hydroxy-4-[4-[2-hydroxy-4-(2-methoxy-1-methyl-2-oxo-ethoxy)phenyl]-6-(4-methoxyphenyl)-1,3,5-triazin-2-yl]phenoxy]propanoate, or a combination of two or more thereof, most preferably the compound of formula (1) according to presently claimed invention is selected from methyl 3-[3-(benzotriazol-2-yl)-5-tert-butyl-4-hydroxy-phenyl]propanoate, ethyl 3-[3-(benzotriazol-2-yl)-5-tert-butyl-4-hydroxy-phenyl]propanoate, 3-[3-(benzotriazol-2-yl)-5-tert-butyl-4-hydroxy-phenyl]propanoic acid, 3-[3-tert-butyl-5-(5-chlorobenzotriazol-2-yl)-4-hydroxy-phenyl]propanoic acid, methyl 3-[3-tert-butyl-5-(5-chlorobenzotriazol-2-yl)-4-hydroxy-phenyl]propanoate, ethyl 3-[3-tert-butyl-5-(5-chlorobenzotriazol-2-yl)-4-hydroxy-phenyl]propanoate, methyl 2-[4-[4,6-bis(2,4-dimethylphenyl)-1,3,5-triazin-2-yl]-3-hydroxy-phenoxy]propanoate, methyl 2-[4-[4,6-bis(4-phenylphenyl)-1,3,5-triazin-2-yl]-3-hydroxy-phenoxy]propanoate, methyl 2-[4-(4,6-diphenyl-1,3,5-triazin-2-yl)-3-hydroxy-phenoxy]propanoate, methyl 2-[4-(4,6-diphenyl-1,3,5-triazin-2-yl)-3-hydroxy-phenoxy]butanoate, methyl 2-[4-[4,6-bis(2,4-dimethylphenyl)-1,3,5-triazin-2-yl]-3-hydroxy-phenoxy]-2-methyl-propanoate, methyl 2-[4-[4,6-bis(2,4-dimethylphenyl)-1,3,5-triazin-2-yl]-3-hydroxy-phenoxy]octanoate, methyl 2-[4-[4,6-bis(2,4-dimethylphenyl)-1,3,5-triazin-2-yl]-3-hydroxy-phenoxy]hexanoate, methyl 2-[4-[4,6-bis(2,4-dihydroxyphenyl)-1,3,5-triazin-2-yl]-3-hydroxy-phenoxy]propanoate, isooctyl 2-[4-[4,6-bis[2-hydroxy-4-(1-methyl-2-octoxy-2-oxo-ethoxy)phenyl]-1,3,5-triazin-2-yl]-3-hydroxy-phenoxy]propanoate (tinuvin 477), isooctyl 2-[4-[4,6-bis(4-phenylphenyl)-1,3,5-triazin-2-yl]-3-hydroxy-phenoxy]propanoate (tinuvin 479), methyl 2-[2-[4-[4,6-bis(2,4-dimethylphenyl)-1,3,5-triazin-2-yl]-3-hydroxy-phenoxy]ethoxy]propanoate, methyl 2-[4-[4,6-bis(4-phenylphenyl)-1,3,5-triazin-2-yl]-3-hydroxy-phenoxy]-3-butoxy-propanoate, methyl 2-[3-hydroxy-4-[4-[2-hydroxy-4-(1-methoxycarbonylpropoxy)phenyl]-6-(4-methoxyphenyl)-1,3,5-triazin-2-yl]phenoxy]butanoate, methyl 2-[4-[4,6-bis(4-butoxy-2-hydroxy-phenyl)-1,3,5-triazin-2-yl]-3-hydroxy-phenoxy]propanoate, methyl 2-[4-[4,6-bis(4-hexoxy-2-hydroxy-3-methyl-phenyl)-1,3,5-triazin-2-yl]-3-hydroxy-2-methyl-phenoxy]propanoate, ethyl 2-[4-[4,6-bis(4-phenylphenyl)-1,3,5-triazin-2-yl]-3-hydroxy-phenoxy]butanoate, methyl 2-[4-[4,6-bis(4-phenylphenyl)-1,3,5-triazin-2-yl]-3-hydroxy-phenoxy]octanoate, methyl 2-[4-[4,6-bis[2-hydroxy-4-(1-methoxycarbonylpropoxy)phenyl]-1,3,5-triazin-2-yl]-3-hydroxy-phenoxy]butanoate, methyl 2-[4-[4,6-bis(2,4-di-methylphenyl)-1,3,5-triazin-2-yl]-3-hydroxy-phenoxy]butanoate, ethyl 2-[4-[4,6-bis(4-phenylphenyl)-1,3,5-triazin-2-yl]-3-hydroxy-phenoxy]propanoate, or a combination of two or more thereof, and in particular, according to presently claimed invention the compound of formula (I) is selected from methyl 3-[3-(benzotriazol-2-yl)-5-tert-butyl-4-hydroxy-phenyl]propanoate, ethyl 3-[3-(benzotriazol-2-yl)-5-tert-butyl-4-hydroxy-phenyl]propanoate, 3-[3-(benzotriazol-2-yl)-5-tert-butyl-4-hydroxy-phenyl]propanoic acid, 3-[3-tert-butyl-5-(5-chlorobenzotriazol-2-yl)-4-hydroxy-phenyl]propanoic acid, methyl 3-[3-tert-butyl-5-(5-chlorobenzotriazol-2-yl)-4-hydroxy-phenyl]propanoate, ethyl 3-[3-tert-butyl-5-(5-chlorobenzotriazol-2-yl)-4-hydroxy-phenyl]propanoate, or a combination of two or more thereof.

In another preferred embodiment, the compound of formula (I) is selected from

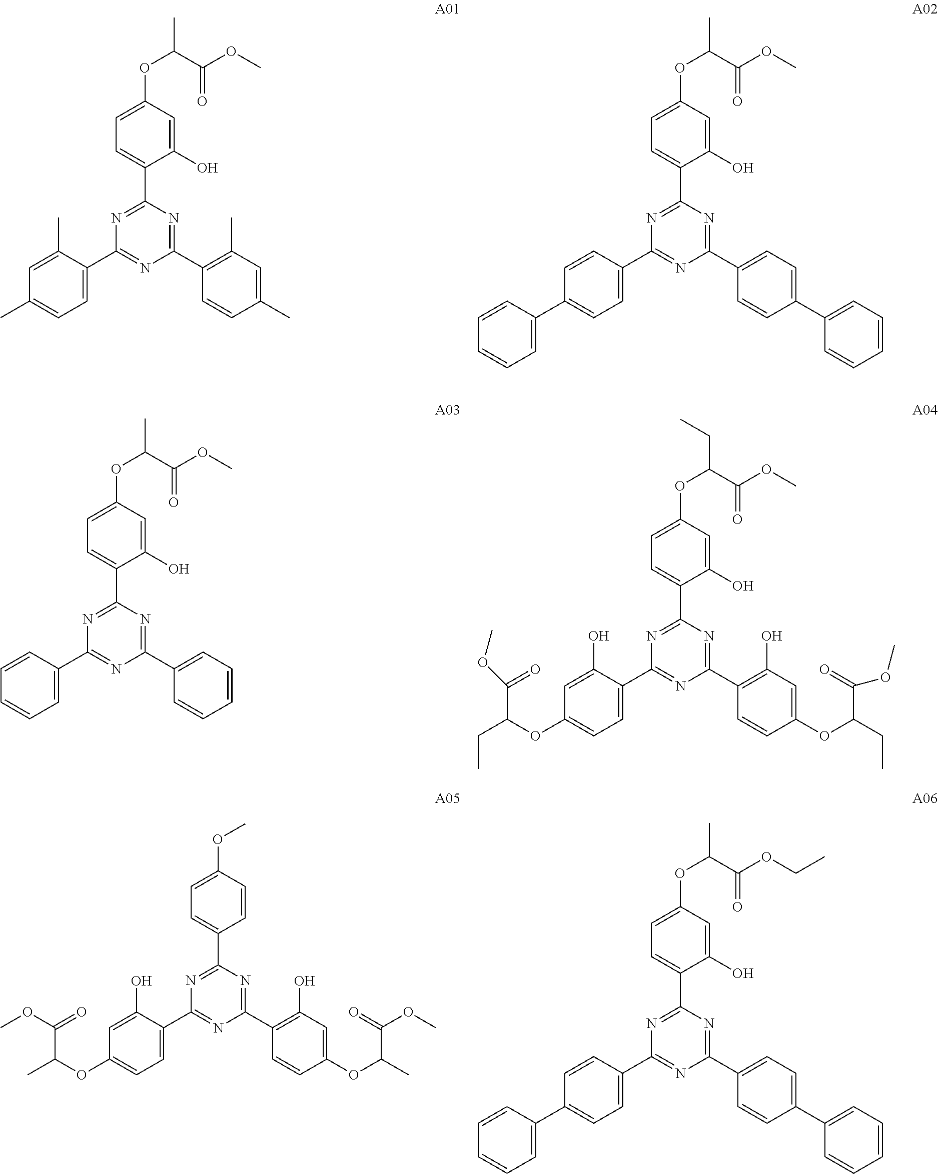

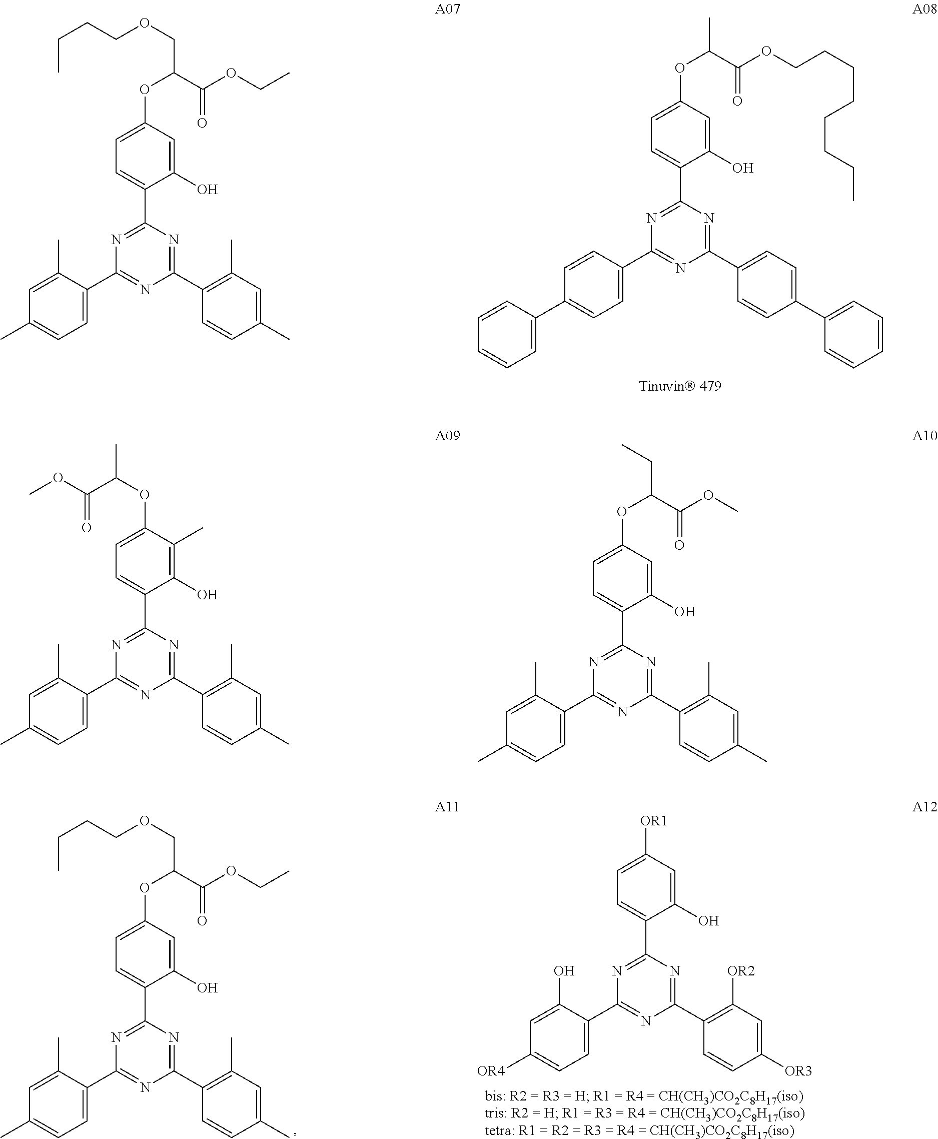

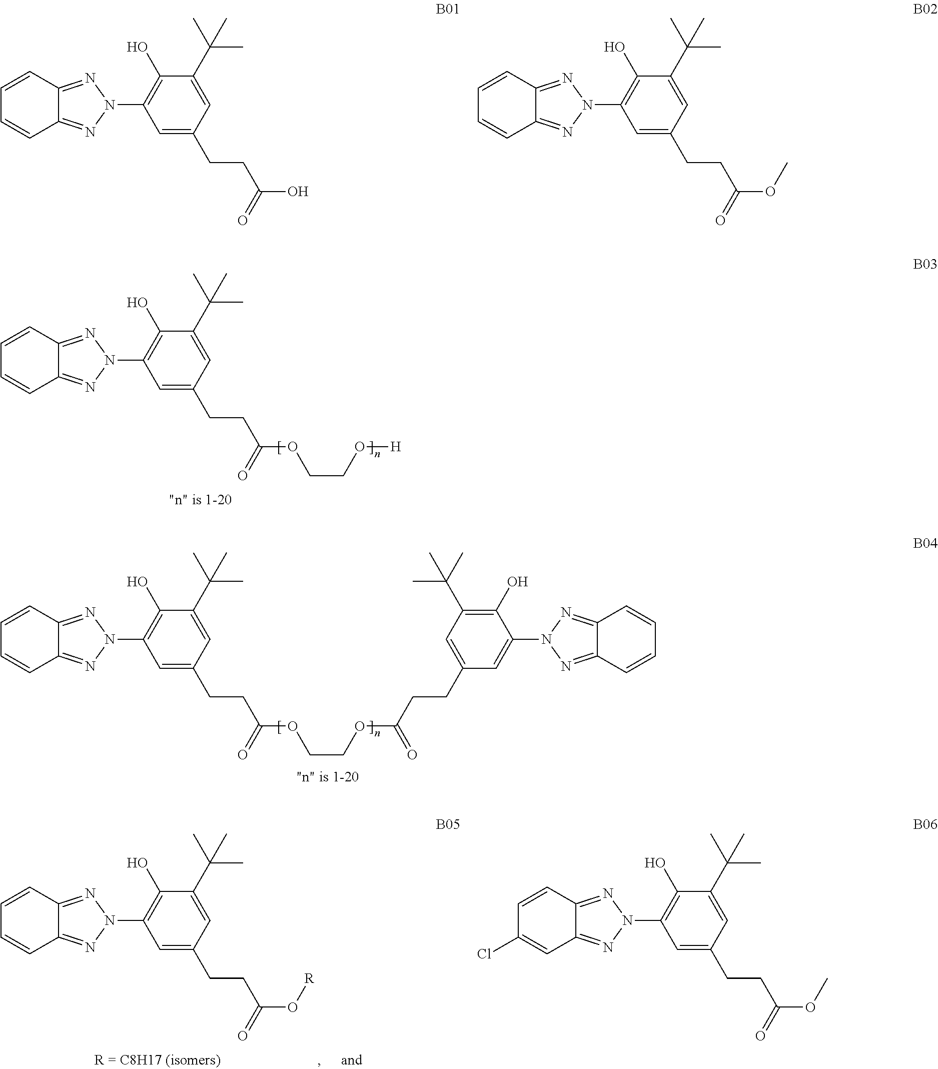

In another preferred embodiment, the compound of formula (II) according to presently claimed invention are selected from pentaerythritol, or trimethylolpropane, substituted or unsubstituted aromatic phenol having one or more hydroxy functional groups, substituted or unsubstituted aralkyl having one or more hydroxy functional groups, substituted or unsubstituted cyclic alcohol having one or more hydroxy functional groups, polyalkyleneoxide having one or more hydroxy functional groups, substituted or unsubstituted polyglycerols, or a combination of two or more thereof.

In another preferred embodiment, the compound of formula (II) according to presently claimed invention has average number of hydroxy groups in the range of 1 to 100, more preferably the polyhydric alcohol according to presently claimed invention has average number of hydroxy groups in the range of 1 to 50, even more preferably the polyhydric alcohol according to presently claimed invention has average number of hydroxy groups in the range of 1 to 30, most preferably the polyhydric alcohol according to presently claimed invention has average number of hydroxy groups in the range of 1 to 20, and in particular preferably the polyhydric alcohol according to presently claimed invention has average number of hydroxy groups in the range of 2 to 10.

In another preferred embodiment, the compound of formula (II) according to presently claimed invention has a hydroxy number calculated in the range of 50 to 5000 mg KOH/g, preferably in the range of 100 to 3000 mg KOH/g, more preferably in the range of 200 to 2500 mg KOH/g, even more preferably in the range of 250 to 2000 mg KOH/g, most preferably in the range of 250 to 1000 mg KOH/g, and in particular in the range of 250 to 800 mg KOH/g.

In another preferred embodiment, the compound of formula (II) according to presently claimed invention has viscosity in the range of 1 to 5000 mPa·S measured at 23° C. according to EN ISO 3219.

In another preferred embodiment, the compound of formula (II) is selected from alkoxylated trimethylolethane, ethoxylated trimethylolethane, propoxylated trimethylolethane, alkoxylated trimethylolpropane, ethoxylated trimethylolpropane, propoxylated trimethylolpropane, alkoxylated 1,1,1-Trimethylolisobutane, ethoxylated 1,1,1-Trimethylolisobutane, propoxylated 1,1,1-Trimethylolisobutane, alkoxylated 1,1,1-Trimethylolpentane, alkoxylated 1,1,1-Tri(hydroxymethyl)-2,2-dimethylpropane, alkoxylated 2-(Hydroxymethyl)-2-(2-methylpropyl)-1,3-propanediol, alkoxylated 2-(Hydroxymethyl)-2-(methoxymethyl)-1,3-propanediol, alkoxylated trimethylolheptane, alkoxylated 2-allyl-2-(hydroxymethyl)propane-1,3-diol, alkoxylated tris(hydroxymethyl)undecane, alkoxylated trimethylolacetic acid, alkoxylated pentaerythritol monoallyl ether, alkoxylated α,α,α-tris(hydroxymethyl)toluene, alkoxylated pentaerythritol, ethoxylated pentaerythritol, propoxylated pentaerythritol, alkoxylated dipentaerythritol, ethoxylated dipentaerythritol, propoxylated dipentaerythritol, alkoxylated tripentaerythritol, ethoxylated tripentaerythritol, propoxylated tripentaerythritol, [3-hydroxy-2,2-bis(hydroxymethyl)propyl]dodecanoate, 3-hydroxy-2,2-bis(hydroxymethyl)propyl octadecenoate, pentaerythritol monoleate, 2-(methoxymethyl)-2-methyl-1,3-propanediol, 2-(2,5-Dioxahexyl)-2-methyl-1,3-propanediol, ditrimethylolpropane, alkoxylated ditrimethylolethane, ethoxylated ditrimethylolethane, pentaerythritol tris(2-hydroxyethyl ether, Diallyl pentaerythritol, trimethylolpropane diallyl ether, tristrimethylolpropane, alkoxylated tristrimethylolpropane, ethoxylated tristrimethylolpropane, propoxylated tristrimethylolpropane, 2-methyl-, 1,1′-[2,2-bis(hydroxymethyl)-1,3-propanediyl]2-propenoic ester.

In another preferred embodiment, the compound of formula (II) is selected from

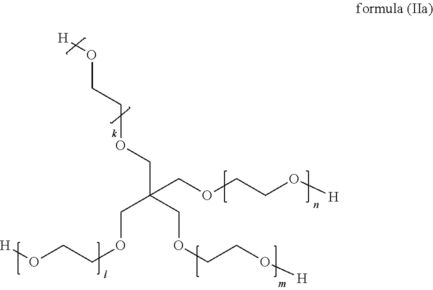

wherein the sum of n+m+k+1=1-30, preferably 1-20, more preferably 4-18 and most preferably 4-14;

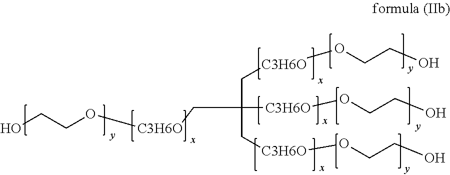

wherein the sum of x+y=1-50, preferably 1-30, more preferably 4-20 and most preferably 4-10;

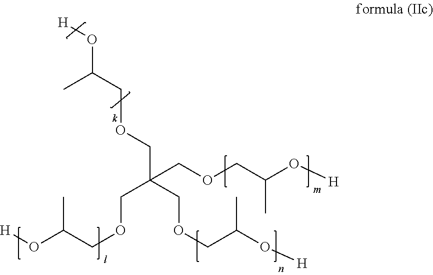

wherein the sum of n+m+k+1=1-30, preferably 1-20, more preferably 4-18 and even more preferably 4-10;

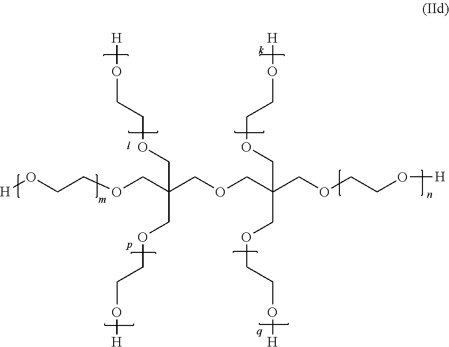

wherein the sum of k+l+n+m+p+q=0-30, preferably 0-20, more preferably 4-20 and even more preferably 6-16; and

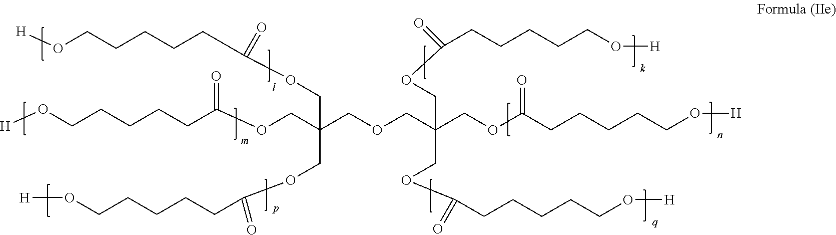

wherein the sum of k+l+n+m+p+q=0-30, preferably 4-20, more preferably 6-16 and even more preferably 6-12.

In another preferred embodiment, the mol ratio of compound of formula (I) to compound of formula (II) is in the range of 1 to 20 to 20 to 1, more preferably the mol ratio of compound of formula (I) to compound of formula (II) is in the range of 1 to 10 to 10 to 1, most preferably the mol ratio of compound of formula (I) to compound of formula (II) is in the range of 1 to 5 to 5 to 1, and in particular preferably the mol ratio of compound of formula (I) to compound of formula (II) is in the range of 1 to 3 to 3 to 1.

In another preferred embodiment, the compound of formula (I) and the compound of formula (II) is reacted in the presence of at least one catalyst,

In another preferred embodiment, the catalyst is selected from Tin compound, Zr compound, Bi compound, Zn compound, or a combination of two or more thereof.

In another preferred embodiment, the Zr compound according to presently claimed invention is selected from ZrO₂, Zirconium halide, Zr(O(O)CR₆₁)₂, Zr(═O)(O(O)CR₆₁)₂, Zr(O(O)CR₆₁)₄, Zr(OCR₆₁)₄, Zr(O(O)CR₆₂C(O)O), or Zr(O(O)CR₆₂C(O)O)₂, Zr(O(O)₂SR₆₁)₂, Zr(O(O)₂SR₆₁)₄, Zr(O(O)₂SR₆₂S(O)₂O), or Zr(O(O)₂SR₆₂S(O)₂O)₂, wherein R₆₁ is selected from substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, substituted or unsubstituted C₁-C₂₄ heteroaryl, substituted or unsubstituted C₇-C₂₄ arylalkyl, substituted or unsubstituted, linear or branched C₁-C₂₄ heteroalkyl, substituted or unsubstituted, linear or branched C₂-C₂₄ heteroalkenyl, substituted or unsubstituted C₅-C₂₄ heterocycloalkyl, or substituted or unsubstituted C₅-C₂₄ heterocycloalkenyl; and R₆₂ is selected from substituted or unsubstituted, linear or branched C₁-C₃₀ alkylene, substituted or unsubstituted, linear or branched 2- to 30-membered hetero alkylene, substituted or unsubstituted, linear or branched C₂-C₂₄ alkenylene, substituted or unsubstituted, linear or branched 3- to 30-membered hetero alkenylene, substituted or unsubstituted C₅-C₂₄ cycloalkylene, or substituted or unsubstituted C₆-C₂₄ arylene.

In another preferred embodiment, the Zn compound according to presently claimed invention is selected from Zinc oxide, Zinc halide, Zn(O(O)CR₆₁)₂, or Zn(O(O)CR₆₂C(O)O), wherein R₆₁ and R₆₂ are as defined as above.

In another preferred embodiment, the Bi compound according to presently claimed invention is selected from Bismuth halide, Bismuth oxide, Bi(R₆₃)₂(O(O)CR₆₁), Bi(R₆₃)(O(O)CR₆₂C(O)O), or Bi(O(O)CR₆₁)₃, Bi(R₆₃)₂(O(O)₂SR₆₁), Bi(OR₆₃)₃, Bi(R₆₃)(O(O)₂SR₆₂S(O)₂O), or Bi(O(O)₂SR₆₁)₃, wherein R₆₁ and R₆₂ are as defined above, and R₆₃ is selected from substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, substituted or unsubstituted C₁-C₂₄ heteroaryl, substituted or unsubstituted C₇-C₂₄ arylalkyl, substituted or unsubstituted, linear or branched C₁-C₂₄ heteroalkyl, substituted or unsubstituted, linear or branched C₂-C₂₄ heteroalkenyl, substituted or unsubstituted C₅-C₂₄ heterocycloalkyl, or substituted or unsubstituted C₅-C₂₄ heterocycloalkenyl.

In another preferred embodiment, the Zr(O(O)CR₆₁)₂, Zr(O)(O(O)CR₆₁)₂, Zr(O(O)CR₆₁)₄, Zr(OCR₆₁)₄, Zr(O(O)CR₆₂C(O)O), Zr(O(O)CR₆₂C(O)O)₂ Zr(O(O)₂SR₆₁)₂, Zr(O(O)₂SR₆₁)₄, Zr(O(O)₂SR₆₂S(O)₂O), or Zr(O(O)₂SR₆₂S(O)₂O)₂, compounds according to presently claimed invention are selected from zirconium acetate, zirconium octoate, zirconium 2-ethylhexanoate, zirconium decanoate, zirconium neodecanoate, bis(acetato-o)oxozirconium, bis(cyclopentadienyl)zirconium bis(trifluoromethanesulfonate) tetrahydrofuran adduct, zirconium(iv) acetylacetonate, zirconium(iv) tetrapropoxide, zirconium(iv) tetrabutoxide, zirconium 3-methyl-3-pentoxide, tetrakis(2-methyl-3-butene-2-oxy)zirconium, or tetrakis(1-methoxy-2-methyl-2-propoxy) zirconium.

In another preferred embodiment, the Zn(O(O)CR₆₁)₂, or Zn(O(O)CR₆₂C(O)O) compounds according to presently claimed invention are selected from zinc neodecanoate, zinc octoate, zinc acetylacetonate, zinc oxalate, zinc acetate, zinc propionate, zinc valerate, zinc pivalate, zinc caprylate, zinc succinate, zinc bis(2-ethylhexanoate), zinc laurate, zinc myristate, zinc bis(tri-fluoroacetate), zinc stearate, zinc citrate, zinc gluconate.

In another preferred embodiment, the Bi(R₆₃)₂(O(O)CR₆₁), Bi(R₆₃)(O(O)CR₆₂C(O)O), Bi(O(O)CR₆₁)₃, Bi(OR₆₃)₃, Bi(R₆₃)₂(O(O)₂SR₆₁), Bi(R₆₃)(O(O)₂SR₆₂S(O)₂O), or Bi(O(O)₂SR₆₁)₃ compounds according to presently claimed invention are selected from bismuth formate, Bismuth octoate, bismuth octanoate, bismuth neodecanoate, bismuth(III) subsalicylate, bismuth neododecanoate, bismuth neooctanoate, bismuth, bismuth trineodecanoate, bismuth triacetate, bismuth tris(2-ethylhexanoate, bismuth triflate, or bismuth beta naphthol.

In another preferred embodiment, the catalyst is a protonic acid. Preferable protonic acid are HCl, sulfuric acid, phosphoric acid, p-toluenesulfonic acid, dodecylbenzene sulfonic acid, camphor sulfonic acid, methane sulfonic acid, benzoic acid, acetic acid or mixtures thereof.

In another preferred embodiment, the catalyst is combination of a metal catalyst as listed above and a protonic acid as listed above.

In another preferred embodiment, wherein the catalyst in the reaction is present in an amount in the range of 0.0001 to 30 wt. % based on total weight of compounds of formula (I), more preferably the catalyst in the reaction is present in a total amount in the range of 0.01 to 10 wt. % based on total weight of compounds of formula (I), even more preferably the catalyst in the reaction is present in a total amount in the range of 0.01 to 5 wt. % based on total weight of compounds of formula (I), most preferably the catalyst in the reaction is present in a total amount in the range of 0.01 to 3.0 wt. % based on total weight of compounds of formula (I), and in particular the catalyst in the reaction is present in a total amount in the range of 0.01 to 1 wt. % based on total weight of compounds of formula (I).

In another preferred embodiment, wherein the catalyst in the reaction is present in an amount in the range of 0.0001 to 2.0 mole equivalent based on compounds of formula (I), more preferably the catalyst in the reaction is present in a total amount in the range of 0.0001 to 1.0 mole equivalent based on compounds of formula (I), even more preferably the catalyst in the reaction is present in a total amount in the range of 0.001 to 1.0 mole equivalent based on compounds of formula (I), most preferably the catalyst in the reaction is present in a total amount in the range of 0.001 to 0.5 mole equivalent based on compounds of formula (I), and in particular the catalyst in the reaction is present in a total amount in the range of 0.001 to 0.1 mole equivalent based on compounds of formula (I).

In another preferred embodiment, the reaction is carried out in presence of a solvent or in the absence of a solvent.

In another preferred embodiment, the solvent is selected from ethers, lactones, carbonates, sulfones, N, N-dimethylformamide, N, N-dimethylacetamide, acetonitrile, dimethylsulfoxide, N-methyl-pyrrolidone, N-ethyl-pyrrolidone, aromatic hydrocarbons, dichloroethane, halogenated aromatic hydrocarbon, or a combination of two or more thereof, more preferably the solvent is selected from ethers, N, N-dimethylformamide, N, N-dimethylacetamide, acetonitrile, dimethylsulfoxide, N-methyl-pyrrolidone, N-ethyl-pyrrolidone, aromatic hydrocarbons, dichloroethane, halogenated aromatic hydrocarbon, or a combination of two or more thereof, even more preferably the solvent is selected from ethers, N, N-dimethylformamide, N-methyl-pyrrolidone, N-ethyl-pyrrolidone, aromatic hydrocarbons, dichloroethane, halogenated aromatic hydrocarbon, or a combination of two or more thereof, most preferably the solvent is selected from ethers, N, N-dimethylformamide, N-methyl-pyrrolidone, N-ethyl-pyrrolidone, aromatic hydrocarbons, halogenated aromatic hydrocarbon, or a combination of two or more thereof, and in particular the solvent is selected from ethers, aromatic hydrocarbons, halogenated aromatic hydrocarbon, or a combination thereof.

In another preferred embodiment, the reaction is carried out in presence of a solvent in an amount in the range of 0.5 to 20 times based of total amount of formula (I), more preferably the reaction is carried out in presence of a solvent in an amount in the range of 0.5 to 10 times based of total amount of formula (I), even more preferably the reaction is carried out in presence of a solvent in an amount in the range of 0.5 to 5.0 times based of total amount of formula (1), most preferably the reaction is carried out in presence of a solvent in an amount in the range of 0.5 to 3.0 times based of total amount of formula (I), and in particular the reaction is carried out in presence of a solvent in an amount in the range of 0.5 to 2.0 times based of total amount of formula (I).

In another preferred embodiment, the reaction is carried out at a temperature in the range of ≥30° C. to ≤250° C., more preferably the reaction is carried out at a temperature in the range of ≥100° C. to ≤220° C., even more preferably the reaction is carried out at a temperature in the range of ≥130° C. to ≤220° C. most preferably the reaction is carried out at a temperature in the range of ≥150° C. to ≤220° C. and in particular the reaction is carried out at a temperature in the range of ≥150° C. to ≤200° C.

In another preferred embodiment, the pH of the reaction is maintained ≤8.0, more preferably the pH of the reaction is maintained in the range of ≥0.0 to ≤8.0, even more preferably the pH of the reaction is maintained in the range of ≥0.0 to ≤4.0, most preferably the pH of the reaction is maintained in the range of ≥0.5 to ≤3.5 and in particular preferably the pH of the reaction is maintained in the range of ≥0.5 to ≤3.0.

Within the context of the presently claimed invention, the term “alkyl”, as used herein, refers to an acyclic saturated aliphatic group, including linear or branched alkyl saturated hydrocarbon radicals, denoted by a general formula CₙH₂ₙ₊₁ and wherein n is the number of carbon atoms such as 1, 2, 3, 4, etc.

In a preferred embodiment, the unsubstituted linear C₁-C₂₄ alkyl is preferably selected from the group consisting of methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl, henicosyl, docosyl, tricosyl and tetracosyl; more preferably selected from the group consisting of hexyl, heptyl, octyl, nonyl, decyl, undecyl, dodecyl, tridecyl, tetradecyl, pentadecyl, hexadecyl, heptadecyl, octadecyl, nonadecyl, eicosyl, henicosyl, docosyl, tricosyl and tetracosyl; even more preferably selected from the group consisting of methyl, ethyl, propyl, butyl, pentyl, hexyl, heptyl, octyl, nonyl, decyl, undecyl, dodecyl, tridecyl, tetradecyl and pentadecyl; most preferably selected from the group consisting of methyl, ethyl, propyl, butyl, pentyl, hexyl, heptyl, octyl, nonyl, decyl, undecyl and dodecyl; and in particular selected from the group consisting of methyl, ethyl, propyl, butyl, pentyl, hexyl, heptyl, octyl, nonyl and decyl.

In a preferred embodiment, the unsubstituted branched C₁-C₂₄ alkyl is preferably selected from the group consisting of isopropyl, iso-butyl, neo-pentyl, 2-ethyl-hexyl, 2-propyl-heptyl, 2-butyl-octyl, 2-pentyl-nonyl, 2-hexyl-decyl, iso-hexyl, iso-heptyl, iso-octyl, iso-nonyl, iso-decyl, iso-dodecyl, iso-tetradecyl, iso-hexadecyl, iso-octadecyl and iso-eicosyl, more preferably selected from the group consisting of 2-ethyl-hexyl, 2-propyl-heptyl, 2-butyl-octyl, 2-pentyl-nonyl, 2-hexyl-decyl, iso-hexyl, iso-heptyl, iso-octyl, iso-nonyl, iso-decyl, iso-dodecyl, iso-tetradecyl, iso-hexadecyl, iso-octadecyl, iso-eicosyl, 2-methyltricosyl, 2-ethyldocosyl, 3-ethylhenicosyl, 3-ethylicosyl, 4-propylhenicosyl, propylnonadecyl, 6-butyldodecyl and 5-ethylundecyl.

In a preferred embodiment, the substituted, linear or branched C₁-C₂₄ alkyl refers to a branched or linear saturated hydrocarbon group having C₁-C₂₄ carbon atoms substituted with functional groups selected from the group consisting of hydroxy, alkoxy, C(═O)—R, CN and SR, wherein R is selected from the group consisting of hydrogen, substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl and substituted or unsubstituted C₇-C₂₄ arylalkyl.

In a preferred embodiment, the substituted, linear or branched C₁-C₂₄ alkyl refers to a branched or linear saturated hydrocarbon group having C₁-C₂₄ carbon atoms substituted with functional groups selected from the group consisting of hydroxy, alkoxy, C(═O)—R, CN and SR, preferably selected from the group consisting of 1-hydroxy methyl, 1-methoxy methyl, 1-hydroxy ethyl, 1-hydroxy propyl, 1-hydroxy butyl, 1-hydroxy pentyl, 1-hydroxy hexyl, 1-hydroxy heptyl, 1-hydroxy octyl, 1-hydroxy nonyl, decyl, 1-hydroxy undecyl, 1-hydroxy dodecyl, 1-hydroxy tridecyl, 1-hydroxy tetradecyl, 1-hydroxy pentadecyl, 1-hydroxy hexadecyl, 1-hydroxy heptadecyl, 1-hydroxy octadecyl, 1-hydroxy nonadecyl, 1-hydroxy eicosyl, 1-hydroxy henicosyl, 1-hydroxy docosyl, 1-hydroxy tricosyl, 1-hydroxy tetracosyl, 1-methoxy methyl, 1-methoxy ethyl, 1-methoxy propyl, 1-methoxy butyl, 1-methoxy pentyl, 1-methoxy hexyl, 1-methoxy heptyl, 1-methoxy octyl, 1-methoxy nonyl, decyl, 1-methoxy undecyl, 1-methoxy dodecyl, 1-methoxy tridecyl, 1-methoxy tetradecyl, 1-methoxy pentadecyl, 1-methoxy hexadecyl, 1-methoxy heptadecyl, 1-methoxy octadecyl, 1-methoxy nonadecyl, 1-methoxy eicosyl, 1-methoxy henicosyl, 1-methoxy docosyl, 1-methoxy tricosyl, 1-methoxy tetracosyl, 2-methoxy propyl, 2-methoxy butyl, 2-methoxy pentyl, 2-methoxy hexyl, 2-methoxy heptyl, 2-methoxy octyl, 2-methoxy nonyl, decyl, 2-methoxy undecyl, 2-methoxy dodecyl, 2-methoxy tridecyl, 2-methoxy tetradecyl, 2-methoxy pentadecyl, 2-methoxy hexadecyl, 2-methoxy heptadecyl, 2-methoxy octadecyl, 2-methoxy nonadecyl, 2-methoxy eicosyl, 2-methoxy henicosyl, 2-methoxy docosyl, 2-methoxy tricosyl, 2-methoxy tetracosyl, 1-acetoxy methyl, 1-acetoxy ethyl, 1-acetoxy propyl, 1-acetoxy butyl, 1-acetoxy pentyl, 1-acetoxy hexyl, 1-acetoxy heptyl, 1-acetoxy octyl, 1-acetoxy nonyl, decyl, 1-acetoxy undecyl, 1-acetoxy dodecyl, 1-acetoxy tridecyl, 1-acetoxy tetradecyl, 1-acetoxy pentadecyl, 1-acetoxy hexadecyl, 1-acetoxy heptadecyl, 1-acetoxy octadecyl, 1-acetoxy nonadecyl, 1-acetoxy eicosyl, 1-acetoxy henicosyl, 1-acetoxy docosyl, 1-acetoxy tricosyl, 1-acetoxy tetracosyl, 1-cyano methyl, 1-cyano ethyl, 1-cyano propyl, 1-cyano butyl, 1-cyano pentyl, 1-cyano hexyl, 1-cyano heptyl, 1-cyano octyl, 1-cyano nonyl, decyl, 1-cyano undecyl, 1-cyano dodecyl, 1-cyano tridecyl, 1-cyano tetradecyl, 1-cyano pentadecyl, 1-cyano hexadecyl, 1-cyano heptadecyl, 1-cyano octadecyl, 1-cyano nonadecyl, 1-cyano eicosyl, 1-cyano henicosyl, 1-cyano docosyl, 1-cyano tricosyl, 1-cyano tetracosyl, 2-cyano propyl, 2-cyano butyl, 2-cyano pentyl, 2-cyano hexyl, 2-cyano heptyl, 2-cyano octyl, 2-cyano nonyl, decyl, 2-cyano undecyl, 2-cyano dodecyl, 2-cyano tridecyl, 2-cyano tetradecyl, 2-cyano pentadecyl, 2-cyano hexadecyl, 2-cyano heptadecyl, 2-cyano octadecyl, 2-cyano nonadecyl, 2-cyano eicosyl, 2-cyano henicosyl, 2-cyano docosyl, 2-cyano tricosyl, 2-cyano tetracosyl, 1-thioyl methyl, 1-thioyl ethyl, 1-thioyl propyl, 1-thioyl butyl, 1-thioyl pentyl, 1-thioyl hexyl, 1-thioyl heptyl, 1-thioyl octyl, 1-thioyl nonyl, decyl, 1-thioyl undecyl, 1-thioyl dodecyl, 1-thioyl tridecyl, 1-thioyl tetradecyl, 1-thioyl pentadecyl, 1-thioyl hexadecyl, 1-thioyl heptadecyl, 1-thioyl octadecyl, 1-thioyl nonadecyl, 1-thioyl eicosyl, 1-thioyl henicosyl, 1-thioyl docosyl, 1-thioyl tricosyl and 1-thioyl tetracosyl.

In a preferred embodiment, the term alkenyl denotes unsubstituted, linear C₂-C₂₄ alkenyl which is preferably selected from the group consisting of 1-propenyl, 1-butenyl, 1-pentenyl, 1-hexenyl, 2-hexenyl, 1-heptenyl, 2-heptenyl, 1-octenyl, 2-octenyl, 1-nonenyl, 2-nonenyl, 1-decenyl, 2-decenyl, 1-undecenyl, 2-undecenyl, 1-dodecenyl, 2-dodecenyl, 1-tridecenyl, 2-tridecenyl, 1-tetradecenyl,2-tetradecenyl, 1-pentadecenyl,2-pentadecenyl, 1-hexadecenyl,2-hexadecenyl, 1-heptadecenyl,2-heptadecenyl, 1-octadecenyl, 2-octadecenyl, 1-nonadecenyl,2-nonadecenyl,1-eicosenyl and 2-eicosenyl, more preferably selected from 1-hexenyl,2-hexenyl, 1-heptenyl, 2-heptenyl, 1-octenyl, 2-octenyl, 1-nonenyl, 2-nonenyl, 1-decenyl, 2-decenyl, 1-undecenyl, 2-undecenyl, 1-dodecenyl, 2-dodecenyl, 1-tridecenyl, 2-tridecenyl, 1-tetradecenyl,2-tetradecenyl, 1-pentadecenyl, 2-pentadecenyl, 1-hexadecenyl, 2-hexadecenyl, 1-heptadecenyl,2-heptadecenyl, 1-octadecenyl,2-octadecenyl, 1-nonadecenyl,2-nonadecenyl, 1-eicosenyl and 2-eicosenyl, 20-henicosenyl, 2-docosenyl, 6-tricosenyl and 2-tetracosenyl.

In a preferred embodiment, the unsubstituted branched C₂-C₂₄ alkenyl is selected from the group consisting of isopropenyl, iso-butenyl, neo-pentenyl, 2-ethyl-hexenyl, 2-propyl-heptenyl, 2-butyl-octenyl, 2-pentyl-nonenyl, 2-hexyl-decenyl, iso-hexenyl, iso-heptenyl, iso-octenyl, iso-nonenyl, iso-decenyl, iso-dodecenyl, iso-tetradecenyl, iso-hexadecenyl, iso-octadecenyl, iso-eicosenyl, 2-methyl tricosenyl, 2-ethyl docosenyl, 3-ethylhenicosenyl, 3-ethyl icosenyl, 4-propylhenicosenyl, 4-propylnonadecenyl, 6-butyldodecenyl, 5-ethylundedcenyl, 1,4-hexadienyl, 1,3-hexadienyl, 2,5-hexadienyl, 3,5-hexadienyl, 2,4-hexadienyl, 1,3,5-hexatrienyl, 1,3,6-hepta-trienyl, 1,4,7-octatrienyl or 2-methyl-1,3,5hexatrienyl, 1,3,5,7-octatetraenyl, 1,3,5,8-nonatetraenyl, 1,4,7,10-undecatetraenyl, 2-ethyl-1,3,6,8-nonatetraenyl, 2-ethenyl-1,3,5,8-nonatetraenyl, 1,3,5,7,9-decapentaenyl, 1,4,6,8,10-undecapentaenyl and 1,4,6,9,11-dodecapentaenyl.

In a preferred embodiment, the substituted, linear or branched C₂-C₂₄ alkenyl refers to a branched or a linear unsaturated hydrocarbon group having C₂-C₂₄ carbon atoms substituted with functional groups selected from, hydroxy, alkoxy, C(═O)—R, CN and SR; wherein R is hydrogen, substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, substituted or unsubstituted C₇-C₂₄ arylalkyl.

In a preferred embodiment the substituted, linear or branched C₂-C₂₄ alkenyl refers to a branched or an linear unsaturated hydrocarbon group having C₂-C₂₄ carbon atoms substituted with functional groups selected from hydroxy, alkoxy, C(═O)—R, CN and SR; preferably selected from the group consisting of 2-hydroxy propenyl, 3-hydroxy butenyl, 3-hydroxy pentenyl, 5-hydroxy hexenyl, 7-hydroxy heptenyl, 3-hydroxy octenyl, 5-hydroxy nonenyl, decyl, 11-hydroxy undecenyl, 9-hydroxy dodecenyl, 6-hydroxy tridecenyl, 4-hydroxy tetradecenyl, 6-hydroxy pentadecenyl, 3-hydroxy hexadecenyl, 2-hydroxy heptadecenyl, 7-hydroxy octadecenyl, 6-hydroxy nonadecenyl, 4-hydroxy eicosenyl, 2-hydroxy henicosenyl, 3-hydroxy docosenyl, 2-hydroxy tricosenyl, 23-hydroxy tetracosenyl, 1-methoxy ethenyl, 2-methoxy propenyl, 4-methoxy butenyl, 3-methoxy pentenyl, 5-methoxy hexenyl, 2-methoxy heptenyl, 5-methoxy octenyl, 3-methoxy nonenyl, 6-methoxy undecenyl, 1-methoxy dodec-2-enyl, 1-methoxy tridec-5-enyl, 3-methoxy tetradic-5-enyl, 3-methoxy pentade-12-encyl, 10-methoxy hexadec-15-enyl, 12-methoxy heptadic-16-enyl,1-methoxy octadec-3-enyl, 1-methoxy nonadec-2-enyl, 1-methoxy eicos-20-enyl, 1-methoxy henicos-2-enyl, 1-methoxy docos-4-enyl, 1-methoxy tricos-22-enyl, 1-methoxy tetra-cos-23-enyl, 2-methoxy prop-1-enyl, 2-methoxy but-1-enyl, 2-methoxy pent-4-enyl, 2-methoxy hex-2-enyl, 2-methoxy hept-3-enyl, 2-methoxy oct-7-enyl, 2-methoxy non-5-enyl, 2-methoxy un-dec-10-enyl, 2-methoxy dodec-4-enyl, 2-methoxy tridec-12-enyl, 2-methoxy tetradic-10-enyl, 2-methoxy pentadec-14-enyl, 2-methoxy hexadec-1-enyl, 2-methoxy heptadic-1-enyl, 2-methoxy octadic-12-enyl, 2-methoxy nonadec-10-enyl, 2-methoxy eicos-18-enyl, 2-methoxy henicos-2-enyl, 2-methoxy docos-3-enyl, 20-methoxy tricos-2-enyl, 21-methoxy tetracos-4-enyl, 1-acetoxy ethenyl, 1-acetoxy prop-1-enyl, 1-acetoxy but-2-enyl, 1-acetoxy pent-4-enyl, 1-acetoxy hex-2-enyl, 1-acetoxy hept-1-enyl, 1-acetoxy oct-7-enyl, 1-acetoxy non-2-enyl, 5-acetoxy dec-3-enyl, 1-acetoxy undec-10-enyl, 1-acetoxy dodec-2-enyl, 1-acetoxy tridec-12-enyl, 10-acetoxy tetradec-2-enyl, 15-acetoxy pentadec-2-enyl, 10-acetoxy hexadec-2-enyl, 11-acetoxy heptadec-1-enyl, 13-acetoxy octadec-2-enyl, 1-acetoxy nonadec-14-enyl, 20-acetoxy eicos-19-enyl, 1-acetoxy henicos-2-enyl, 1-acetoxy docos-10-enyl, 1-acetoxy tricos-22-enyl, 1-acetoxy tetracos-23-enyl, 1-cyano eth-1-enyl, 1-cyano prop-2-enyl, 1-cyano but-2-enyl, 1-cyano pent-3-enyl, 1-cyano hex-5-enyl, 1-cyano hept-6-enyl, 1-cyano oct-2-enyl, 1-cyano non-3-enyl, 11-cyano un-dec-2-enyl, 10-cyano dodec-2-enyl, 10-cyano tridec-12-enyl, 1-cyano tetradec-3-enyl, 1-cyano pentadec-14-enyl, 1-cyano hexadec-15-enyl, 1-cyano heptadec-2-enyl, 1-cyano octadec-3-enyl, 1-cyano nonadec-18-enyl, 1-cyano eicos-10-enyl, 1-cyano henicos-20-enyl, 15-cyano docos-3-enyl, 1-cyano tricos-20-enyl, 1-cyano tetracos-2-enyl, 2-cyano prop-2-enyl, 2-cyano but-1-enyl, 2-cyano pent-1-enyl, 2-cyano hex-3-enyl, 2-cyano hept-6-enyl, 2-cyano oct-1-enyl, 2-cyano non-8-enyl, 2-cyano undec-10-enyl, 2-cyano dodec-1-enyl, 2-cyano tridec-12-enyl, 2-cyano tetradec-10-enyl, 2-cyano pentadec-3-enyl, 2-cyano hexadec-2-enyl, 2-cyano heptadec-1-enyl, 2-cyano octadec-12-enyl, 2-cyano nonadec-15-enyl, 2-cyano eicos-1-enyl, 2-cyano henicos-5-enyl, 2-cyano docos-20-enyl, 2-cyano tricos-22-enyl, 2-cyano tetracos-20-enyl, 1-thionyl eth-1-enyl, 1-thionyl prop-2-enyl, 1-thionyl but-2-enyl, 1-thionyl pent-4-enyl, 1-thionyl hex-2-enyl, 1-thionyl hept-5-enyl, 1-thionyl oct-3-enyl, 1-thionyl non-5-enyl, 1-thionyl undec-10-enyl, 1-thionyl dodec-11-enyl, 1-thionyl tridec-2-enyl, 1-thionyl tetradec-4-enyl, 1-thionyl pentadec-5-enyl, 1-thionyl hexadec-3-enyl, 1-thionyl heptadec-2-enyl, 1-thionyl octadec-3-enyl, 1-thionyl nonadec-15-enyl, 1-thionyl eicos-18-enyl, 1-thionyl henicos-20-enyl, 1-thionyl docos-21-enyl, 1-thionyl tri-cos-20-enyl and 1-thionyl tetracos-22-enyl.

In a preferred embodiment, the substituted or unsubstituted C₅-C₂₄ cycloalkyl refers to a monocyclic and bicyclic 5- to 24-membered saturated cycloaliphatic radical. Representative examples of unsubstituted or branched C₅-C₂₄ monocyclic and bicyclic cycloalkyl include, but are not limited to, cyclopentyl, cyclohexyl, cycloheptyl, cyclooctyl, bicyclo[2.2.1]heptyl, and bicyclo[3.1.1]heptyl.

In another preferred embodiment, the C₅-C₂₄ monocyclic and bicyclic cycloalkyl can be further branched with one or more equal or different alkyl groups such as methyl, ethyl, n-propyl, iso-propyl, n-butyl, iso-butyl, n-pentyl, iso-pentyl, neo-pentyl etc. The representative examples of branched C₃-C₁₀ monocyclic and bicyclic cycloalkyl include, but are not limited to, methyl cyclohexyl and dimethyl cyclohexyl.

In a preferred embodiment, the unsubstituted or substituted C₅-C₂₄ cycloalkenyl refers to a monocyclic and bicyclic 5- to 24-membered unsaturated cycloaliphatic radical which comprises one or more double bonds. Representative examples of C₅-C₂₄ cycloalkenyl include, but are not limited to, cyclopentenyl, cyclohexenyl, cycloheptenyl, cyclooctenyl, cyclononenyl or cyclodecenyl.

These radicals can be branched with one or more equal or different alkyl radical, preferably with methyl, ethyl, n-propyl or iso-propyl. The representative examples of branched C₅-C₂₄ monocyclic and bicyclic cycloalkenyl include, but are not limited to, methyl cyclohexenyl and dimethyl cyclohexenyl.

In a preferred embodiment, the substituted or unsubstituted C₆-C₂₄ aryl may have more than one aromatic ring. The representative examples for substituted and unsubstituted C₆-C₂₄ aryl include phenyl, naphthyl, anthracenyl, tetraphenyl, phenalenyl and phenanthrenyl.

In a preferred embodiment, the arylalkyl refers to an aryl ring attached to an alkyl chain. The representative examples for the arylalkyl include, but are not limited to, 1-phenylmethyl, 1-phenylethyl, 1-phenylpropyl, 1-phenylbutyl, 1-methyl-1-phenyl-propyl, 3-phenylpropyl, 4-phenylbutyl, 3-phenylbutyl and 2-methyl-3-phenyl-propyl.

In a preferred embodiment, the substituted C₆-C₂₄ aryl refers to an aromatic ring having substitution at different positions. The C₆-C₂₄ aryl may have more than one aromatic ring. The representative examples for substituted and unsubstituted C₆-C₂₄ aryl include tolyl, xylyl, 2-hydroxy-phenyl, 2,3-dihydroxyphenyl, 2-methoxy phenyl, 2-hydroxy-4-methoxyphenyl, 2,4-dimethoxy-phenyl, 2-chlorophenyl, 2-chloro-4-hydroxyphenyl, 2-chloro-4-methoxy phenyl, 3-chloro-4-methoxyphenyl, 2-methyl-4-methoxy-6-chlorophenyl and 2-acetyl-4-hydroxyphenyl.

Within the context of the presently claimed invention, the term alkylene refers to an acyclic saturated hydrocarbon chains, which combine different moieties.

The alkylene refers to linear unsubstituted C₁ to C₃₀ carbon atoms, selected from, but not limited to, —CH₂—, —CH₂—CH₂—, —CH₂—CH₂—CH₂—, —CH₂—CH₂—CH₂—CH₂—, —CH₂—CH₂—CH₂—CH₂—CH₂—, —CH₂—CH₂—CH₂—CH₂—CH₂—CH₂—, —CH₂—CH₂—CH₂—CH₂—CH₂—CH₂—CH₂—, —CH₂—CH₂—CH₂—CH₂—CH₂—CH₂—CH₂—CH₂—, —CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—, or —CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—. In another preferred embodiment the branched unsubstituted alkylene selected from, but not limited to, —CH₂—C(CH₃)H—, —CH₂—C(CH₃)H—CH₂—, —CH₂—CH₂—C(CH₃)H—CH₂—, —C(CH₃)₂—CH₂—CH₂—C(CH₃)H—CH₂—, —CH₂—C(CH₃)H—CH₂—CH₂—CH₂—CH₂—, —CH₂—C(CH₃)H—CH₂—CH₂—CH₂—CH₂—CH₂—, —CH₂—CH₂—CH₂—CH₂—CH₂—CH₂—CH₂—C(CH₃)H—, —C(CH₃)H—CH₂—CH₂—CH₂—CH₂—CH₂—CH₂—CH₂—CH₂—, —C(CH₃)H—CH₂—CH₂—CH₂—CH₂—CH₂—CH₂—CH₂—CH₂—CH₂—, —C(CH₃)₂—CH₂—CH₂—CH₂—CH₂—CH₂—CH₂—CH₂—CH₂—CH₂—CH₂—, —CH₂—C(CH₃)₂—CH₂—CH₂—CH₂—CH₂—CH₂—CH₂—CH₂—CH₂—CH₂—CH₂—, —CH₂—C(CH₃)H—CH₂—CH₂—CH₂—CH₂—CH₂—CH₂—CH₂—CH₂—CH₂—CH₂—CH₂—, —C(CH₃)₂—CH₂—CH₂—CH₂—CH₂—CH₂—CH₂—CH₂—CH₂—CH₂—CH₂—CH₂—CH₂—CH₂—, —CH₂—CH₂—CH₂—C(CH₃)H—CH₂—CH₂—CH₂—CH₂—CH₂—CH₂—CH₂—CH₂—CH₂—CH₂—CH₂—, —C(CH₃)H—CH₂—CH₂—CH₂—CH₂—CH₂—CH₂—CH₂—CH₂—CH₂—CH₂—CH₂—CH₂—CH₂—CH₂—CH₂—, —CH₂—C(CH₃)H—CH₂—CH₂—CH₂—CH₂—CH₂—CH₂—CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—CH₂—CH₂—CH₂—CH₂—CH₂—CH₂—CH₂—C(CH₃)H—, —C(CH₃)H—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—, —C(CH₃)H—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—, —C(CH₃)H—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—, —C(CH₃)H—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—, —C(CH₃)H—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—, or —C(CH₃)H—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—CH₂—.

In another preferred embodiment the product obtained according to presently claimed invention has weight average molecular weight in the range of 500 to 20′000, preferably in the range of 800 to 10′000, more preferred in the range of 1′000 to 8′000, even more preferred in the range of 1′000 to 5′000 as determined by GPC using polystyrene as internal standard.

In another preferred embodiment the product obtained according to presently claimed invention has solubility is greater than 10 g per 100 mL xylene at room temperature, more preferably the product obtained according to presently claimed invention has solubility is greater than 20 g per 100 mL xylene at room temperature, most preferably the product obtained according to presently claimed invention has solubility is greater than 30 g per 100 mL xylene at room temperature, and in particular preferably the product obtained according to presently claimed invention has solubility is greater than 40 g per 100 mL xylene at room temperature.

In another preferred embodiment the presently claimed invention is directed to the use of the product or a salt thereof according to presently claimed invention as ultraviolet stabilizer.

In another preferred embodiment presently claimed invention is directed to a composition comprising a product or a salt thereof obtained according to presently claimed invention.

In another preferred embodiment presently claimed invention is directed to a method of protecting a material or coating from light, wherein the method comprises a step of providing the product or salt thereof obtained according to presently claimed invention as UV stabilizer. Said product or salt thereof is noted to provide long term stability comparable with commonly used industry standard UV absorber (for e.g., hydroxyphenyl benzotriazole class absorber).

In another preferred embodiment presently claimed invention the composition may be solvent based or water based. Typical examples of organic solvents are aliphatic, aromatic or cycloaliphatic hydrocarbons, alcohols, glycols, esters, acetates and ketones.

In another preferred embodiment presently claimed invention the coatings are surface coatings. Preferably, the composition is an automotive coating composition.

The coating composition is preferably a laquer, in particular a stoving laquer which is used for coating automobiles (automobile finishing lacquers), for example stoving lacquers comprising alkyd/melamine resins and alkyd/acrylic/melamine resins (see H. Wagner and H. F. Sarx, “Lack-kunstharze” (1977), pages 99-123), epoxy/carboxy resins, isocyanate crosslinked acrylic polyols or polyester polyols. Other crosslinking agents include glycoluril resinsor blocked isocyanates.

In another preferred embodiment, the coating composition preferably contains 0.01-10 parts by weight, especially 0.05-10 parts by weight, more especially 0.1-5 parts by weight, of the product, or a salt thereof according to the invention per 100 parts by weight of a solid binder. The binders may in principle be any binders that are customary in the art, for example those described in Ullmann's Encyclopedia of Industrial Chemistry, 5th ed., Vol. A18, pp. 368-426, VCH, Weinheim 1991. The binder will generally be a film-forming binder, based on a thermoplastic or thermosetting resin, predominantly on a thermosetting resin. Examples thereof are alkyd, acrylic, polyester, phenolic, melamine, epoxy and polyurethane resins and mixtures thereof.

It may be a cold-curable or a hot-curable binder, and the addition of a curing catalyst may be advantageous. Suitable catalysts, which accelerate full curing of the binder, are described, for example, in Ullmann's Encyclopedia of Industrial Chemistry, Vol. A18, p. 469, VCH Verlagsgesellschaft, Weinheim 1991.

Multilayer systems are possible here as well, it being possible for the concentration of the stabilisers in the top layer to be higher, for example from 1 to 15 parts by weight, especially from 3 to 10 parts by weight, based on 100 parts by weight of solid binder.

### EMBODIMENTS

In the following, there is provided a list of embodiments to further illustrate the present disclosure without intending to limit the disclosure to the specific embodiments listed below.

1. A product, or a salt thereof obtainable by a process comprising at least the step of:
a) reacting at least one compound of formula (I),

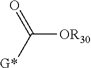

compound of formula (I)
wherein G* is selected from formula (A), or formula (B)

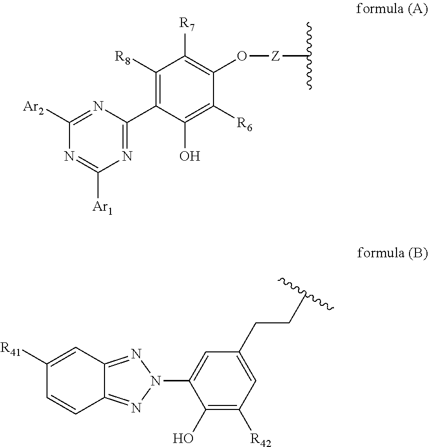

wherein Z is selected from substituted or unsubstituted, linear or branched C₁-C₃₀ alkylene, substituted or unsubstituted, linear or branched 2- to 30-membered hetero alkylene, substituted or unsubstituted, linear or branched C₂-C₂₄ alkenylene, substituted or unsubstituted, linear or branched 3- to 30-membered hetero alkenylene, substituted or unsubstituted C₅-C₂₄ cycloalkylene, or substituted or unsubstituted C₆-C₂₄ arylene,
Ar₁ and Ar₂ are independently of each other a moiety of the formula (C),

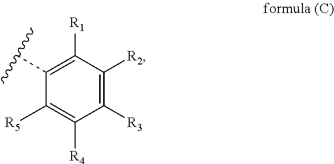

wherein the dotted line is a single bond between formula (C) and the triazinyl ring of formula (A), and R₁, R₂, R₃, R₄ and R₅ are independently of each other selected from hydrogen, substituted or unsubstituted, linear or branched C₁-C₂₄ alkyl, substituted or unsubstituted C₆-C₂₄ aryl, —OH, —OC₁-C₂₄ alkyl; or a moiety of formula (M),

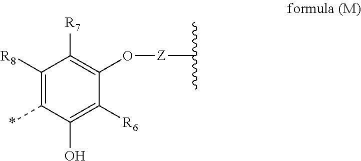

wherein the dotted line is a single bond between formula (M) and the triazinyl ring of formula (A), Z is defined as above, and Z is attached to —C(═O)OR30 and wherein R30 is defined as above,
R6, R7 and R8 are independently of each other selected from hydrogen, or substituted or unsubstituted, linear or branched C1-C24 alkyl;
R41, and R42 independently of each other, are selected from hydrogen, halogen, substituted or unsubstituted, linear or branched C1-C24 alkyl, substituted or unsubstituted, linear or branched C2-C24 alkenyl, substituted or unsubstituted C5-C24 cycloalkyl, substituted or unsubstituted C5-C24 cycloalkenyl, substituted or unsubstituted C6-C24 aryl, or substituted or unsubstituted C7-C24 arylalkyl,
R₃₀ is selected from hydrogen, substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, substituted or unsubstituted C₇-C₂₄ arylalkyl, or —S(═O)₂R₃₁,
wherein R₃₁ is selected from substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₆-C₂₄ aryl, substituted or unsubstituted C₇-C₂₄ arylalkyl,
b) with at least one compound of formula (II)

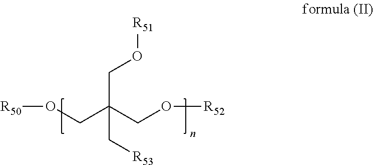

wherein n is an integer from 1 to 20,
wherein R₅₀, R₅₁, and R₅₂ are independently from each other selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, —(C═O)R₅₅, or —[CH₂CHR₅₆O]ₘ—R₅₇;
R₅₃ is selected from hydrogen, substituted or unsubstituted, linear or branched C₁-C₂₄ alkyl, substituted or unsubstituted, linear or branched C₂-C₂₄ alkenyl, or OR₅₉,
wherein R₅₉ is selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, —(C═O)R₅₅, or —[CH₂CHR₅₆O]ₘ—R₅₇,
R₅₄ is selected from hydrogen, substituted or unsubstituted, linear or branched C₁-C₂₄ alkyl, substituted or unsubstituted, linear or branched C₂-C₂₄ alkenyl, or —(C═O)—C₁-C₂₄ alkyl;
R₅₅ is selected from substituted or unsubstituted, linear or branched C₁-C₂₄ alkyl, or substituted or unsubstituted, linear or branched C₂-C₂₄ alkenyl;
R₅₆ is selected from hydrogen, or substituted or unsubstituted, linear or branched C₁-C₂₄ alkyl,
R₅₇ is selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, or —(C═O)R₅₅;
wherein t is an integer from 1 to 20,
wherein j is an integer from 1 to 20,
wherein m is an integer from 1 to 100,
with the proviso that at least one of R₅₀, R₅₁, R₅₂, R₅₄, R₅₇ and R₅₉ is hydrogen; and if n=1, at least one of R₅₀, R₅₁ and R₅₂ is not hydrogen.

2. The product, or a salt thereof according to embodiment 1,
wherein G* is

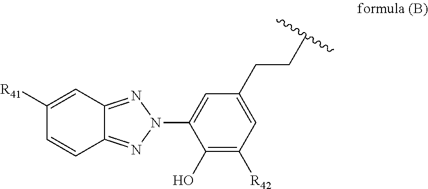

R₄₁, and R₄₂ independently of each other, are selected from hydrogen, halogen, substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, or substituted or unsubstituted C₇-C₂₄ arylalkyl.

3. The product, or a salt thereof according to embodiment 2, wherein R₄₁, and R₄₂ independently of each other, are selected from hydrogen, halogen, substituted or unsubstituted, linear or branched C₁-C₂₄ alkyl, or substituted or unsubstituted, linear or branched C₂-C₂₄ alkenyl.

4. The product, or a salt thereof according to any one of the embodiments 1 to 3, wherein at least one compound of formula (II) is

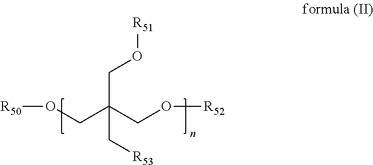

wherein n is an integer from 1 to 10;
wherein R₅₀, R₅₁, and R₅₂ are independently from each other selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, —(C═O)R₅₅, or —[CH₂CHR₅₆O]ₘ—R₅₇;
R₅₃ is selected from hydrogen, substituted or unsubstituted, linear or branched C₁-C₂₄ alkyl, substituted or unsubstituted, linear or branched C₂-C₂₄ alkenyl, or OR₅₉,
wherein R₅₉ is selected from hydrogen, substituted or unsubstituted, linear or branched C₁-C₂₄ alkyl, substituted or unsubstituted, linear or branched C₂-C₂₄ alkenyl, —[(C═O)—(CH₂—)ₜ—NH]ⱼ—R₅₄, —[(C═O)—(CH₂)ₜ—O]ⱼ—R₅₄, —(C═O)R₅₅, or —[CH₂CHR₅₆O]ₘ—R₅₇,
R₅₄ is selected from hydrogen, substituted or unsubstituted, linear or branched C₁-C₂₄ alkyl, or —(C═O)—C₁₋₂₄ alkyl;
R₅₅ is substituted or unsubstituted, linear or branched C₁-C₂₄ alkyl;
R56 is selected from H, or substituted or unsubstituted, linear or branched C₁-C₂₄ alkyl,
R57 is selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]j—R54, —[(C═O)—(CH2)t—O]j—R54, or —(C═O)R55;
wherein t is an integer from 1 to 10,
wherein j is an integer from 1 to 10,
wherein m is an integer from 1 to 50,
with the proviso that at least one of R50, R51, R52, R54 and R57 is hydrogen; and if n=1, at least one of R50, R51 and R52 is not hydrogen.

5. The product, or a salt thereof according to embodiment 4, wherein n is an integer from 2 to 5;
wherein R₅₀, R₅₁, and R₅₂ are independently from each other selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, —(C═O)R₅₅, or —[CH₂CHR₅₆O]ₘ—R₅₇;
R₅₃ is selected from hydrogen, substituted or unsubstituted, linear or branched C₁₋₆ alkyl, substituted or unsubstituted, linear or branched C₂₋₆ alkenyl, or OR₅₉,
wherein R₅₉ is selected from hydrogen, substituted or unsubstituted, linear or branched C₁-C₂₄ alkyl, substituted or unsubstituted, linear or branched C₂-C₂₄ alkenyl, —[(C═O)—(CH₂—)ₜ—NH]ⱼ—R₅₄, —[(C═O)—(CH₂)ₜ—O]ⱼ—R₅₄, —(C═O)R₅₅, or —[CH₂CHR₅₆O]ₘ—R₅₇,
R₅₄ is selected from hydrogen, or substituted or unsubstituted, linear or branched C₁-C₂₄ alkyl;
R₅₅ is substituted or unsubstituted, linear or branched C₁-C₂₄ alkyl;
R₅₆ is selected from H, or substituted or unsubstituted, linear or branched C₁₋₆ alkyl,
R₅₇ is selected from hydrogen, substituted or unsubstituted, linear or branched C₁-C₂₄, —[(C═O)—(CH2)t-O]j-R54, or —(C═O)R55;
wherein t is an integer from 1 to 5,
wherein j is an integer from 1 to 5,
wherein m is an integer from 1 to 20, with the proviso that at least one of R50, R51, R52, R54 and R57 is hydrogen; and if n=1, at least one of R₅₀, R₅₁ and R₅₂ is not hydrogen.

6. The product, or a salt thereof according to embodiment 5, wherein n is an integer from 2 to 3;
wherein R₅₀, R₅₁, and R₅₂ are independently from each other selected from hydrogen, substituted or unsubstituted, linear or branched C₁-C₂₄ alkyl, or —[(C═O)—(CH₂)ₜ—O]ⱼ—R₅₄;
R₅₃ is selected from hydrogen, C₁ alkyl, or OR₅₉, wherein R₅₉ is as defined as above,
R₅₄ is selected from hydrogen, or substituted or unsubstituted, linear or branched C₁_₆ alkyl;
R₅₅ is substituted or unsubstituted, linear or branched C₁₋₆ alkyl;
R₅₆ is selected from H, or C₁ alkyl,
R₅₇ is selected from hydrogen, substituted or unsubstituted, linear or branched C₁-C₂₄ alkyl, or —[(C═O)—(CH₂)ₜ—O]ⱼ—R₅₄,
wherein t is an integer from 3 to 5,
wherein j is an integer from 1 to 2,
wherein m is an integer from 4 to 16,
with the proviso that at least one of R₅₀, R₅₁, R₅₂, R₅₄ and R₅₇ is hydrogen; and if n=1, at least one of R₅₀, R₅₁ and R₅₂ is not hydrogen.

7. The product, or a salt thereof according to any one of the embodiments 1 to 6, wherein the compound of formula (II) has a hydroxy number calculated in the range of 50 to 5000 mg KOH/g.

8. The product, or a salt thereof according to any one of the embodiments 1 to 7, wherein the compound of formula (II) has viscosity in the range of 1 to 5000 mPa·S measured at 23° C. according to EN ISO 3219.

9. The product, or a salt thereof according to any one of the embodiments 1 to 8, wherein the compound of formula (I) is selected from methyl 3-[3-(benzotriazol-2-yl)-5-tert-butyl-4-hydroxy-phenyl]propanoate, ethyl 3-[3-(benzotriazol-2-yl)-5-tert-butyl-4-hydroxy-phenyl]propanoate, 3-[3-(benzotriazol-2-yl)-5-tert-butyl-4-hydroxy-phenyl]propanoic acid, 3-[3-tert-butyl-5-(5-chlorobenzotriazol-2-yl)-4-hydroxy-phenyl]propanoic acid, methyl 3-[3-tert-butyl-5-(5-chlorobenzotriazol-2-yl)-4-hydroxy-phenyl]propanoate, ethyl 3-[3-tert-butyl-5-(5-chlorobenzotriazol-2-yl)-4-hydroxy-phenyl]propanoate, methyl 2-[4-[4,6-bis(2,4-dimethylphenyl)-1,3,5-triazin-2-yl]-3-hydroxy-phenoxy]propanoate, methyl 2-[4-[4,6-bis(4-phenylphenyl)-1,3,5-triazin-2-yl]-3-hydroxy-phenoxy]propanoate, methyl 2-[4-(4,6-diphenyl-1,3,5-triazin-2-yl)-3-hydroxy-phenoxy]propanoate, ethyl 2-[4-[4,6-bis(2,4-dimethylphenyl)-1,3,5-triazin-2-yl]-3-hydroxy-phenoxy]-3-butoxy-propanoate, ethyl 2-[4-(4,6-diphenyl-1,3,5-triazin-2-yl)-3-hydroxy-phenoxy]octanoate, methyl 2-[4-(4,6-diphenyl-1,3,5-triazin-2-yl)-3-hydroxy-phenoxy]butanoate, methyl 2-[4-[4,6-bis(2,4-dimethylphenyl)-1,3,5-triazin-2-yl]-3-hydroxy-phenoxy]-2-methyl-propanoate, methyl 2-[4-[4,6-bis(2,4-dimethylphenyl)-1,3,5-triazin-2-yl]-3-hydroxy-phenoxy]octanoate, methyl 2-[4-[4,6-bis(2,4-dimethylphenyl)-1,3,5-triazin-2-yl]-3-hydroxy-phenoxy]hexanoate, methyl 2-[4-[4,6-bis(2,4-dihydroxyphenyl)-1,3,5-triazin-2-yl]-3-hydroxy-phenoxy]propanoate, octyl 2-[4-[4,6-bis[2-hydroxy-4-(1-methyl-2-octoxy-2-oxo-ethoxy)phenyl]-1,3,5-triazin-2-yl]-3-hydroxy-phenoxy]propanoate (tinuvin 477), 6-methylheptyl 2-[4-[4,6-bis(4-phenylphenyl)-1,3,5-triazin-2-yl]-3-hydroxy-phenoxy]propanoate (tinuvin 479), octyl 2-[4-[4,6-bis(4-phenylphenyl)-1,3,5-triazin-2-yl]-3-hydroxy-phenoxy]propanoate, methyl 2-[2-[4-[4,6-bis(2,4-dimethylphenyl)-1,3,5-triazin-2-yl]-3-hydroxy-phenoxy]ethoxy]propanoate, methyl 2-[4-[4,6-bis(4-phenylphenyl)-1,3,5-triazin-2-yl]-3-hydroxy-phenoxy]-3-butoxy-propanoate, methyl 2-[3-hydroxy-4-[4-[2-hydroxy-4-(1-methoxycarbonylpropoxy)phenyl]-6-(4-methoxyphenyl)-1,3,5-triazin-2-yl]phenoxy]butanoate, methyl 2-[4-[4,6-bis(4-butoxy-2-hydroxy-phenyl)-1,3,5-triazin-2-yl]-3-hydroxy-phenoxy]propanoate, methyl 2-[4-[4,6-bis(4-hexoxy-2-hydroxy-3-methyl-phenyl)-1,3,5-triazin-2-yl]-3-hydroxy-2-methyl-phenoxy]propanoate, ethyl 2-[4-[4,6-bis(4-phenylphenyl)-1,3,5-triazin-2-yl]-3-hydroxy-phenoxy]butanoate, methyl 2-[4-[4,6-bis(4-phenylphenyl)-1,3,5-triazin-2-yl]-3-hydroxy-phenoxy]octanoate, methyl 2-[4-[4,6-bis[2-hydroxy-4-(1-methoxycarbonylpropoxy)phenyl]-1,3,5-triazin-2-yl]-3-hydroxy-phenoxy]butanoate, methyl 2-[4-[4,6-bis(2,4-dimethylphenyl)-1,3,5-triazin-2-yl]-3-hydroxy-phenoxy]butanoate, ethyl 2-[4-[4,6-bis(4-phenylphenyl)-1,3,5-triazin-2-yl]-3-hydroxy-phenoxy]propanoate, methyl 2-[4-[4,6-bis[2-hydroxy-4-(2-methoxy-1-methyl-2-oxo-ethoxy)phenyl]-1,3,5-triazin-2-yl]-3-hydroxy-phenoxy]propanoate, methyl 2-[3-hydroxy-4-[4-[2-hydroxy-4-(2-methoxy-1-methyl-2-oxo-ethoxy)phenyl]-6-(4-methoxyphenyl)-1,3,5-triazin-2-yl]phenoxy]propanoate, or a combination of two or more thereof.

10. The product, or a salt thereof according to embodiment 9, wherein the compound of formula (1) is selected from methyl 3-[3-(benzotriazol-2-yl)-5-tert-butyl-4-hydroxy-phenyl]propanoate, ethyl 3-[3-(benzotriazol-2-yl)-5-tert-butyl-4-hydroxy-phenyl]propanoate, 3-[3-(benzotriazol-2-yl)-5-tert-butyl-4-hydroxy-phenyl]propanoic acid, 3-[3-tert-butyl-5-(5-chloro-benzotriazol-2-yl)-4-hydroxy-phenyl]propanoic acid, methyl 3-[3-tert-butyl-5-(5-chlorobenzotriazol-2-yl)-4-hydroxy-phenyl]propanoate, ethyl 3-[3-tert-butyl-5-(5-chlorobenzotriazol-2-yl)-4-hydroxy-phenyl]propanoate, or a combination of two or more thereof.

11. The product, or a salt thereof according to any one of the embodiments 1 to 10, wherein the mol ratio of compound of formula (I) to compound of formula (II) is in the range of 1 to 20 to 20 to 1.

12. The product, or a salt thereof according to embodiment 11, wherein the mol ratio of compound of formula (I) to compound of formula (II) is in the range of 1 to 5 to 5 to 1.

13. The product, or a salt thereof according to any one of the embodiments 1 to 12, wherein the compound of formula (II) is selected from alkoxylated trimethylolethane, ethoxylated trimethylolethane, propoxylated trimethylolethane, alkoxylated trimethylolpropane, ethoxylated trimethylolpropane, propoxylated trimethylolpropane, alkoxylated 1,1,1-Trimethylolisobutane, ethoxylated 1,1,1-Trimethylolisobutane, propoxylated 1,1,1-Trimethylolisobutane, alkoxylated 1,1,1-Trimethylolpentane, alkoxylated 1,1,1-Tri(hydroxymethyl)-2,2-dimethylpropane, alkoxylated 2-(Hydroxymethyl)-2-(2-methylpropyl)-1,3-propanediol, alkoxylated 2-(Hydroxymethyl)-2-(methoxymethyl)-1,3-propanediol, alkoxylated trimethylolheptane, alkoxylated 2-allyl-2-(hydroxymethyl)propane-1,3-diol, alkoxylated tris(hydroxymethyl)undecane, alkoxylated trimethylolacetic acid, alkoxylated pentaerythritol monoallyl ether, alkoxylated α,α,α-tris(hydroxymethyl)toluene, alkoxylated pentaerythritol, ethoxylated pentaerythritol, propoxylated pentaerythritol, alkoxylated dipentaerythritol, ethoxylated dipentaerythritol, propoxylated dipentaerythritol, alkoxylated tripentaerythritol, ethoxylated tripentaerythritol, propoxylated tripentaerythritol, [3-hydroxy-2,2-bis(hydroxymethyl)propyl]dodecanoate, 3-hydroxy-2,2-bis(hydroxymethyl)propyl octadecenoate, pentaerythritol monoleate, 2-(methoxymethyl)-2-methyl-1,3-propanediol, 2-(2,5-Dioxahexyl)-2-methyl-1,3-propanediol, ditrimethylolpropane, alkoxylated ditrimethylolethane, ethoxylated ditrimethylolethane, pentaerythritol tris(2-hydroxyethyl ether, Diallyl pentaerythritol, trimethylolpropane diallyl ether, tristrimethylolpropane, alkoxylated tristrimethylolpropane, ethoxylated tristrimethylolpropane, propoxylated tristrimethylolpropane, 2-methyl-, 1,1′-[2,2-bis(hydroxymethyl)-1,3-propanediyl]2-propenoic ester.

14. The product, or a salt thereof according to any one of the embodiments 1 to 13 has weight average molecular weight in the range of 500 to 20′000.

15. The product, or a salt thereof according to any one of the embodiments 1 to 14 has solubility is greater than 10 g per 100 mL xylene at room temperature.

16. Use of the product or a salt thereof according to any one of the embodiments 1 to 15 as ultraviolet stabilizer.

17. A composition comprising a product or a salt thereof according to any one of the embodiments 1 to 15.

18. A method of protecting a material or coating from light, wherein the method comprises a step of providing the product according to any one of the embodiments 1 to 15 or salt thereof as UV stabilizer.

The presently claimed invention is illustrated in detail by non-restrictive working examples which follow. More particularly, the test methods specified hereinafter are part of the general disclosure of the application and are not restricted to the specific working examples.

### Materials and Methods

Methyl 3-[3-(benzotriazol-2-yl)-5-tert-butyl-4-hydroxy-phenyl]propanoate is available from Alfa Chemistry, USA.

Tripentaerythritol is available from Sigma-Aldrich, Germany.

Tin (II) 2-ethylhexanoate is available from Sigma-Aldrich, Germany.

ε-Caprolactone is available from Aldrich, Germany.

Polyol 4290, Polyol 4360, Polyol R4630 and Polyol R6405 are available from Perstorp Specialty Chemicals AB, Sweden.

Dibutyltin oxide is available from Sigma-Aldrich, Germany.

Triazine methyl ester compound of formula A01 and triazine methyl ester compound of formula A02 were synthesized analogously to a procedure described in example A1 on page 44 to 45 in patent application GB 2312210 A.

Pernil® ME V HD (coconut-/palmkernel-/palm oil fatty acid methyl ester) is available from BASF, Germany.

Pernil® ME V 05 (oleic acid methyl ester) is available from BASF, Germany.

### Example 1

Tripentaerythritol (75.0 grams, 0.20 mole), tin (II) 2-ethylhexanoate (0.07 grams), and compound of formula B02 (212.0 grams, 0.60 mole, 3 eq) were charged to a reaction flask equipped with an blade impeller, thermometer and attached to a vacuum distillation apparatus equipped with Liebig condenser and receiving flask. Under an argon blanket, the reactor contents were heated to 205° C. and a 20 mbar vacuum was applied. After 2 h reaction time HPLC analysis indicated complete conversion of compound of formula B02. The reactor contents were discharged and cooled yielding 246 grams of a UV absorbing polymer as a greenish tinted melt which solid-ified on cooling.

### Example 2

Tripentaerythritol (55.9 grams, 0.15 mole), ε-caprolactone (137.0 grams, 1.20 mole) and tin (II) 2-ethylhexanoate (0.06 grams) were charged to a reaction flask equipped with an blade impeller, thermometer and attached to a vacuum distillation apparatus equipped with Liebig condenser and receiving flask. Under an argon blanket, the reactor contents were heated to 170° C. After stirring for 2 h at 170° C. compound of formula B02 (159.0 grams, 0.45 mole) was charged into the reactor. The reactor contents were heated up to 205° C. and a 20 mbar vacuum was applied. The reactor contents were stirred for 4 h at 205° C. and 20 mbar. After cooling down to 50° C. acetone (500.0 grams), Tonsil Optimum FF (9.0 grams) and concentrated sulfuric acid (2.0 grams) were added and kept overnight at room temperature. The mixture was then filtered and the filtrate concentrated at 50° C. and 25 mbar at a rotary evaporator yielding 340 grams of the UV absorbing polymer as a brownish, highly viscous liquid.

### Example 3

A 100 mL glass flask containing a magnetic stir bar was placed in an agitating heating block. ε-caprolactone (2.30 grams, 0.020 mol), tin (II) 2-ethylhexanoate (0.02 grams) and tripentaerythritol (3.70 grams, 0.01 mole) were transferred into the flask. Under an argon flow, the flask content was heated to 170° C. and a 20 mbar vacuum was applied. After 2 h stirring the reactor contents were heated up to 180° C. and compound of formula B02 (10.60 grams, 0.03 moles) was added. A 205° C. heating and a 20 mbar vacuum were applied for 4 hours. A HPLC analysis indicated a conversion of >99% of compound of formula B02. The flask contents were discharged, cooled and ground yielding 10.0 grams of the UV absorbing polymer as a beige powder with a melting point of 86° C.

### Example 4

A 100 mL glass flask containing a magnetic stir bar was placed in an agitating heating block. ε-caprolactone (2.30 grams, 0.020 mol), tin (II) 2-ethylhexanoate (0.02 grams) and tripentaerythritol (3.70 grams, 0.01 mole) were transferred into the flask. Under an argon flow, the flask content was heated to 170° C. for 30 min. After applying a 20 mbar vacuum stirring was continued for 2 h yielding a colorless suspension. Compound of formula B02 (14.10 grams, 0.04 moles) was added. The flask contents were heated at 170° C. for 2 h, at 205° C. for 3 h and finally at 225° C. for 3 h. A HPLC analysis indicated a conversion of >99% of compound of formula B02. The flask contents were discharged, cooled and ground yielding 15.1 grams of the UV absorbing polymer as a beige powder with a melting point of 71° C.

### Example 5

A 100 mL glass flask containing a magnetic stir bar was placed in an agitating heating block. Polyol 4290 (20 grams, 0.025) was transferred into the flask. The flask was placed under vacuum for four times, then backfilled with argon. Under an argon flow, the flask content was heated to 180° C. Dibutyltin oxide (52.6 milligrams) and benzotriazole methyl ester of formula B02 (30.9 grams, 0.088 mole) were added to the flask. The flask was placed under vacuum. The vacuum was reduced to 25 mbar within 4 hours and stirring was continued for 6 hours. An HPLC analysis indicated a complete conversion of the benzotriazole methyl ester of formula B02. The flask contents were discharged and cooled yielding 46.2 grams of the UV absorbing polymer as a dark yellow, highly viscous melt.

All experiments were conducted in accordance with example 5 with varying polyols as depicted in the following table.

| | | | Benzotriazol | | | |
|---|---|---|---|---|---|---|
| | Polyol | | methyl ester | DBTO | UV absorbing polymer product | |
| Example | Trade | Amount | Amount | Amount | Yield | |
| # | name | [grams] | [grams] | [milligrams] | [grams] | Aspect |
| 5 | Polyol | 20.0 | 30.9 | 52.6 | 46.2 | dark yellow, highly viscous |
| | 4290 | | | | | melt |
| 6 | | 18.4 | 32.5 | 47.6 | 46.1 | dark yellow, highly viscous |
| | | | | | | melt |
| 7 | Polyol | 18.3 | 32.8 | 61.0 | 45.0 | brownish, highly viscous |
| | 4360 | | | | | melt |
| 8 | | 15.8 | 35.3 | 54.4 | 44.8 | brownish, highly viscous |
| | | | | | | melt |

### Example 9

A 100 mL glass flask containing a magnetic stir bar was placed in an agitating heating block. Polyol R4630 (13.5 grams) was transferred into the flask. The flask was placed under vacuum for four times, then backfilled with argon. Under an argon flow, the flask content was heated to 140° C. Dibutyltin oxide (81.4 milligrams) and benzotriazole methyl ester of formula B02 (40.3 grams, 0.114 mole) were added to the flask. The flask was placed under vacuum. The vacuum was reduced 25 mbar within 6 hours while increasing heating temperature to 175° C. Stirring was continued for 21 hours while increasing the heating temperature to 200° C. Polyol R4630 (0.61 grams, 0.002 mole) was added to the flask and stirring continued for 13 hours. An HPLC analysis indicated a conversion 0f >99% of the benzotriazole methyl ester of formula B02. The flask contents were discharged and cooled yielding 39.2 grams of the UV absorbing polymer as a brownish, highly viscous melt.

All experiments were conducted in accordance with example 9 with varying polyols as depicted in the following table.

| | | | Benzotriazol | | | |
|---|---|---|---|---|---|---|
| | Polyol | | methyl ester | DBTO | UV absorbing polymer product | |
| Example | Trade | Amount | Amount | Amount | Yield | |
| # | name | [grams] | [grams] | [milligrams] | [grams] | Aspect |
| 9 | Polyol | 13.5 + 0.61 | 40.3 | 81.4 | 39.2 | brownish, highly viscous |
| | R4630 | | | | | melt |
| 10 | | 11.0 + 2.05 | 43.8 | 68.2 | 49.8 | brownish, highly viscous |
| | | | | | | melt |
| 11 | Polyol | 17.4 | 37.1 | 47.3 | 38.6 | brownish, highly viscous |
| | R6405 | | | | | melt |
| 12 | | 15.4 + 6.6 | 39.2 | 53.8 | 49.2 | brownish, highly viscous |
| | | | | | | melt |

### Example 13

A 100 mL glass flask containing a magnetic stir bar was placed in an agitating heating block. Polyol R6405 (18.7 grams, 0.023 mol) was transferred into the flask. The flask was placed under vacuum for four times, then backfilled with argon. Applying a vacuum of 20 mbar, the flask content was heated to 170-180° C. After backfilling argon, dibutyltin dilaurate (76.3 milligrams) and Pernil ME V HD (1.28 grams) were added to the flask and stirring continued for 3 hours. Benzotriazole methyl ester of formula B02 (30.2 grams, 0.086 mole) was added to the flask. After applying a 20 mbar vacuum, stirring was continued for 10 hours. An HPLC analysis indicated a conversion of more than 98% of the benzotriazole methyl ester of formula B02. The flask contents were discharged and cooled yielding 44.9 grams of the UV absorbing polymer as a brownish melt which solidifies on cooling.

All experiments were conducted in accordance with example 13 with varying amounts of Pernil ME V HD as depicted in the following table.

| | Polyol | Benzotriazol | Pernil ME | Dibutyltin | | |
|---|---|---|---|---|---|---|
| | R6405 | methyl ester | V HD | dilaurate | UV absorbing polymer product | |
| Example | Amount | Amount | Amount | Amount | Yield | |
| # | [grams] | [grams] | [grams] | [milligrams] | [grams] | Aspect |
| 13 | 18.7 | 30.2 | 1.26 | 68.2 | 44.9 | brownish melt |
| 14 | 18.7 | 31.8 | 1.26 | 68.2 | 46.7 | brownish melt |
| 15 | 18.7 | 30.2 | 2.52 | 68.2 | 46.1 | brownish melt |

### Example 16

A 100 mL glass flask containing a magnetic stir bar was placed in an agitating heating block. Polyol 4360 (19.5 grams, 0.031 mole) was transferred into the flask. The flask was placed under vacuum for four times, then backfilled with argon. Applying a vacuum of 20 mbar, the flask content was heated to 170-180° C. After backfilling argon, dibutyltin dilaurate (99.0 milligrams) and Pernil ME V HD (1.77 grams) were added to the flask and stirring continued for 2 hours at 75 mbar. Benzotriazole methyl ester of formula B02 (30.7 grams, 0.087 mole) was added to the flask. After applying a 20 mbar vacuum, stirring was continued for 5 hours. Polyol 4360 (6.72 grams, 0.01 mole) was added to the flask and stirring was continued for 8 hours. Dibutyltin dilaurate (57.5 milligrams) was added to the flask and stirring continued for 8 hours. Polyol 4360 (1.0 grams, 0.002 mole) was added to the flask and stirring was continued for 8 hours. An HPLC analysis indicated a conversion of 97% of the benzotriazole methyl ester of formula B02. The flask contents were discharged and cooled yielding 54.2 grams of the UV absorbing polymer as a light orange melt which solidifies on cooling.

All experiments were conducted in accordance with example 16 with varying amounts of the raw materials as depicted in the following table.

| | Polyol | Benzotriazol | Pernil ME | Dibutyltin | | |
|---|---|---|---|---|---|---|
| | R6405 | methyl ester | V HD | dilaurate | UV absorbing polymer product | |
| Example | Amount | Amount | Amount | Amount | Yield | |
| # | [grams] | [grams] | [grams] | [milligrams] | [grams] | Aspect |
| 16 | 19.5 + | 30.7 | 1.77 | 99.0 + 57.5 | 54.2 | light orange melt |
| | 6.72 + | | | | | |
| | 1.00 | | | | | |
| 17 | 18.9 + | 29.7 | 3.36 | 97.1 + 59.2 | 52.7 | light orange melt |
| | 7.82 + | | | | | |
| | 1.00 | | | | | |
| 18 | 17.6 + | 31.6 | 1.57 | 89.3 + 56.3 | 52.2 | light orange melt |
| | 7.35 + | | | | | |
| | 1.00 | | | | | |
| 19 | 17.3 + | 31.1 | 3.10 | 89.9 + 57.2 | 53.7 | light orange melt |
| | 7.81 + | | | | | |
| | 1.00 | | | | | |

### Example 20

A 100 mL glass flask containing a magnetic stir bar was placed in an agitating heating block. Polyol R6405 (18.3 grams) was transferred into the flask. The flask was placed under vacuum for four times, then backfilled with argon. Applying a vacuum of 20 mbar, the flask content was heated to 170-180° C. After backfilling argon, dibutyltin dilaurate (66.7 milligrams) and Pernil ME V 05 (1.24 grams) were added to the flask and stirring continued for 2 hours at 75 mbar. Benzotriazole methyl ester of formula B02 (29.6 grams, 0.084 mole) was added to the flask. After applying a 20 mbar vacuum, stirring was continued for 9 hours. An HPLC analysis indicated a conversion of 99% of the benzotriazole methyl ester of formula B02. The flask contents were discharged and cooled yielding 44.0 grams of the UV absorbing polymer as a brownish melt which solidifies on cooling.

All experiments were conducted in accordance with example 20 with varying amounts of the raw materials as depicted in the following table.

| | Polyol | Benzotriazol | Pernil ME | Dibutyltin | UV absorbing polymer | |
|---|---|---|---|---|---|---|
| | R6405 | methyl ester | V 05 | dilaurate | product | |
| Example | Amount | Amount | Amount | Amount | Yield | |
| # | [grams] | [grams] | [grams] | [milligrams] | [grams] | Aspect |
| 20 | 18.3 | 29.6 | 1.24 | 66.7 | 44.0 | brownish |
| | | | | | | melt |
| 21*) | 18.3 | 28.0 | 2.47 | 66.1 | 43.3 | brownish |
| | | | | | | melt |
| 22*) | 18.3 | 31.1 | 1.23 | 71.7 | 43.9 | brownish |
| | | | | | | melt |
| 23*) | 18.3 | 29.6 | 2.46 | 69.7 | 44.2 | brownish |
| | | | | | | melt |

*)in the experiments marked with *) the flask contents were heated for 16 hours at 180° C. and 20 mbar.

### Example 24

A 100 mL glass flask containing a magnetic stir bar was placed in an agitating heating block. Polyol R6405 (16.6 grams) was transferred into the flask. The flask was placed under vacuum, then backfilled with argon for four times. Applying a vacuum of 20 mbar, the flask content was heated to 170-180° C. Dibutyltin dilaurate (66.6 milligrams) and ε-caprolactone (1.20 grams, 0.010 mol) were added to the flask and stirring continued for 2 hours at 75 mbar. Then benzotriazole methyl ester of formula B02 (33.9 grams, 0.096 mole) were added to the flask. After applying a 20 mbar vacuum, stirring was continued for 26 hours. An HPLC analysis indicated a conversion of 97.7% of the benzotriazole methyl ester of formula B02. The flask contents were discharged and cooled yielding 44.3 grams of the UV absorbing polymer as a brownish melt which solidifies on cooling.

All experiments were conducted in accordance with example 24 with varying polyols as depicted in the following table.

| | | | Benzotriazol | ε-caprolac- | Dibutyltin | UV absorbing | |
|---|---|---|---|---|---|---|---|
| | Polyol | | methyl ester | tone | dilaurate | polymer product | |
| Example | Trade | Amount | Amount | Amount | Amount | Yield | |
| # | name | [grams] | [grams] | [grams] | [milligrams] | [grams] | Aspect |
| 24 | Polyol | 16.6 | 33.9 | 1.20 | 66.6 | 44.3 | brownish |
| | R6405 | | | | | | melt |
| 25 | | 16.6 | 33.9 | 2.38 | 60.8 | 47.0 | brownish |
| | | | | | | | melt |
| 26 | Polyol | 20.0 | 28.3 | 1.48 | 80.5 | 45.0 | brownish |
| | 4290 | | | | | | melt |
| 27 | | 19.9 | 30.9 | 1.35 | 74.8 | 47.3 | brownish |
| | | | | | | | melt |

### Example 28

A 100 mL glass flask containing a magnetic stir bar was placed in an agitating heating block. ε-caprolactone (1.70 grams, 0.015 mol), tin (II) 2-ethylhexanoate (0.02 grams) and tripentaerythritol (2.80 grams, 0.008 mole) were transferred into the flask. Under an argon flow, the flask content was heated to 170° C. for 30 min. After applying a 20 mbar vacuum stirring was continued for 2 hours. Then the heating temperature was increased to 205° C. and triazine methyl ester compound of formula A01 (10.9 grams, 0.023 moles) was added. The flask contents were heated at 205° C. for 2 h at 20 mbar. A HPLC analysis indicated a conversion of >99% of compound of formula A01. The flask contents were discharged, cooled and ground yielding 10.5 grams of the UV absorbing triazine polymer as a beige powder with a melting point of 107° C.

### Example 29

A 100 mL glass flask containing a magnetic stir bar was placed in an agitating heating block. ε-caprolactone (2.80 grams, 0.025 mole), tin (II) 2-ethylhexanoate (0.02 grams) and trimethylolpropane (3.40 grams, 0.025 mole) were transferred into the flask. Under an argon flow, the flask content was heated to 170° C. for 30 min. After applying a 20 mbar vacuum stirring was continued for 2 hours. Then Benzotriazole methyl ester of formula B02 (17.70 g, 0.050 mole) was added and the heating temperature was increased to 205° C. The flask contents were heated at 205° C. for 6 hours at 20 mbar. A HPLC analysis indicated a conversion of >99% of compound of formula B02. The flask contents were discharged, cooled and ground yielding 19.1 grams of the UV absorbing benzotriazole polymer as a colorless powder with a melting point of 55° C.

### Example 30

A 100 mL glass flask containing a magnetic stir bar was placed in an agitating heating block. Ditrimethylolpropane (5.00 grams, 0.02 mole), tin (II) 2-ethylhexanoate (0.02 grams) and Pernil ME V 05 (5.90 grams, ca. 0.02 mole) were transferred into the flask. Under an argon flow, the flask content was heated to 170° C. After applying a 20 mbar vacuum stirring was continued for 2 hours and then for 2 hours at 205° C. Then Benzotriazole methyl ester of formula B02 (14.10 g, 0.04 mole) was added. The flask contents were heated at 205° C. for 20 hours at 20 mbar. A HPLC analysis indicated a conversion of >95% of compound of formula B02. The flask contents were discharged, cooled and ground yielding 19 grams of the UV absorbing benzotriazole polymer as a light yellow, highly viscous substance with a residual amount of compound of formula B02 of 2.7%.

### Example 31

A 100 mL glass flask containing a magnetic stir bar was placed in an agitating heating block. Ditrimethylolpropane (5.00 grams, 0.02 mole), tin (II) 2-ethylhexanoate (0.02 grams) and ε-caprolactone (2.30 grams, 0.02 mole) were transferred into the flask. Under an argon flow, the flask content was heated to 170° C. After applying a 20 mbar vacuum stirring was continued for 2 hours. Then Benzotriazole methyl ester of formula B02 (21.20 g, 0.06 mole) was added. The flask content was heated at 205° C. for 5 hours at 20 mbar and then discharged, cooled and ground yielding 33 grams of the UV absorbing benzotriazole polymer as a light yellow powder with a melting point of 64° C. and with a residual amount of compound of formula B02 of 1.2%.

### Example 32

Example 32 was performed according to example 32 but using 4.60 grams (0.04 mole) of ε-caprolactone. The UV absorbing polymer product was yielded as yellowish powder (35 grams, melting point: 54° C., 1.1% residual amounts of compound of formula B02).

### Comparative Example C3

Comparative example C3 was prepared according to a procedure described in WO2010/130752 on page 26. The product was obtained as a colorless powder, which is less than 1% soluble in xylene [1H NMR (CDCl3): δ=11.8 (s, 1H), 8.12 (d, 1H), 7.81-7.93 (m 2H), 7.42-7.48 (m, 2H), 7.20 (d, 1H), 4.12-4-17 (m, 2H), 2.94 (t, 2H), 2.69 (t, 2H), 1.52 (s, 9H)].

| | Absorbance A | | | | | | |
|---|---|---|---|---|---|---|---|
| | in dichloro- | | | | | | |
| | methane at 20 mg/L | | | Mn | Mw | Solubility at | |
| | at | at | at | (GPC) | (GPC) | room temperature [g] | |
| | 320 | 340 | 360 | [Da] | [Da] | xylene | toluene |
| Example | nm | nm | nm | Da | Da | (g) | (g) |
| Example 1 | 0.59 | 0.72 | 0.54 | 1037 | 1323 | >50 | n.d. |
| Example 2 | 0.35 | 0.43 | 0.33 | 2141 | 3294 | >50 | n.d. |
| Example 3 | 0.59 | 0.71 | 0.53 | 1331 | 1708 | >10 | n.d. |
| Example 4 | 0.47 | 0.57 | 0.44 | 1634 | 1946 | n.d. | n.d. |
| Example 5 | 0.50 | 0.60 | 0.46 | 1284 | 1616 | n.d. | 85 |
| Example 6 | 0.40 | 0.48 | 0.37 | 1228 | 1649 | n.d. | 85 |
| Example 7 | 0.67 | 0.81 | 0.62 | 1066 | 1348 | n.d. | 75 |
| Example 8 | 0.42 | 0.51 | 0.39 | 892 | 1323 | n.d. | 75 |
| Example 9 | 0.70 | 0.85 | 0.64 | 1039 | 1157 | n.d. | 65 |
| Example 10 | 0.47 | 0.57 | 0.44 | 1067 | 1265 | n.d. | 65 |
| Example 11 | 0.59 | 0.72 | 0.55 | 1348 | 1671 | n.d. | 60 |
| Example 12 | 0.71 | 0.86 | 0.66 | 1289 | 1629 | n.d. | 65 |
| Example 13 | 0.44 | 0.54 | 0.41 | 1258 | 1612 | 65 | 75 |
| Example 14 | 0.42 | 0.51 | 0.39 | 1277 | 1646 | 65 | 75 |
| Example 15 | 0.36 | 0.44 | 0.33 | 1288 | 1663 | 65 | 75 |
| Example 16 | 0.46 | 0.56 | 0.43 | 1111 | 1272 | 85 | n.d. |
| Example 17 | 0.34 | 0.42 | 0.32 | 1118 | 1266 | 85 | n.d. |
| Example 18 | 0.50 | 0.60 | 0.46 | 1135 | 1276 | 85 | n.d. |
| Example 19 | 0.39 | 0.47 | 0.36 | 1125 | 1289 | 85 | n.d. |
| Example 20 | 0.36 | 0.43 | 0.33 | 1302 | 1638 | 85 | n.d. |
| Example 21 | 0.44 | 0.53 | 0.40 | 1298 | 1653 | 85 | n.d. |
| Example 22 | 0.37 | 0.45 | 0.34 | 1335 | 1665 | 85 | n.d. |
| Example 23 | 0.49 | 0.59 | 0.45 | 1329 | 1680 | 85 | n.d. |
| Example 24 | 0.49 | 0.60 | 0.45 | 1382 | 1813 | 85 | n.d. |
| Example 25 | 0.55 | 0.67 | 0.51 | 1397 | 1842 | 85 | n.d. |
| Example 26 | 0.36 | 0.45 | 0.34 | 1461 | 1679 | 85 | n.d. |
| Example 27 | 0.40 | 0.49 | 0.37 | 1336 | 1680 | 85 | n.d. |
| Example 28 | 0.61 | 0.59 | 0.18 | n.d. | n.d. | >10 | >10 |
| Example 29 | 0.59 | 0.72 | 0.54 | 776 | 887 | 85 | n.d. |
| Example 30 | 0.57 | 0.69 | 0.53 | 953 | 1155 | 85 | n.d. |
| Example 31 | 0.54 | 0.65 | 0.50 | 1000 | 1129 | 85 | n.d. |
| Example 32 | 0.51 | 0.62 | 0.47 | 1125 | 1305 | 85 | n.d. |
| C1 | | | | | | 1.5 | |
| C3 | | | | | | 2 | |

1.-18. (canceled)

19. A product, or a salt thereof obtained by a process comprising the step of:
a) reacting at least one compound of formula (I), 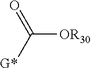
compound of formula (I)
wherein G* is selected from formula (A), or formula (B) 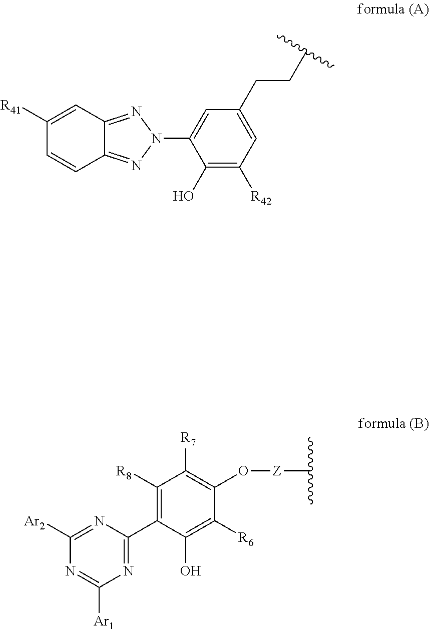
wherein Z is selected from substituted or unsubstituted, linear or branched C₁-C₃₀ alkylene, substituted or unsubstituted, linear or branched 2- to 30-membered hetero alkylene, substituted or unsubstituted, linear or branched C₂-C₂₄ alkenylene, substituted or unsubstituted, linear or branched 3- to 30-membered hetero alkenylene, substituted or unsubstituted C₅-C₂₄ cycloalkylene, or substituted or unsubstituted C₆-C₂₄ arylene, Ar₁ and Ar₂ are independently of each other a moiety of the formula (C), 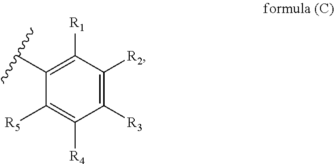
wherein the dotted line is a single bond between formula (C) and the triazinyl ring of formula (A), and R₁, R₂, R₃, R₄ and R₅ are independently of each other selected from hydrogen, substituted or unsubstituted, linear or branched C₁-C₂₄ alkyl, substituted or unsubstituted C₆-C₂₄ aryl, —OH, —OC₁-C₂₄ alkyl; or a moiety of formula (M), 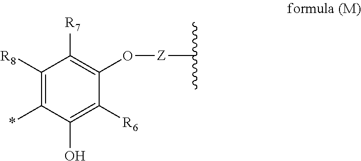
wherein the dotted line is a single bond between formula (M) and the triazinyl ring of formula (A), Z is defined as above, and Z is attached to —C(═O)OR₃₀ and wherein R₃₀ is defined as above,
R₆, R₇ and R₈ are independently of each other selected from hydrogen, or substituted or unsubstituted, linear or branched C₁-C₂₄ alkyl;
R₄₁, and R₄₂ independently of each other, are selected from hydrogen, halogen, substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, or substituted or unsubstituted C₇-C₂₄ arylalkyl,
R₃₀ is selected from hydrogen, substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, substituted or unsubstituted C₇-C₂₄ arylalkyl, or —S(═O)₂R₃₁, wherein R₃₁ is selected from substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₆-C₂₄ aryl, substituted or unsubstituted C₇-C₂₄ arylalkyl,
b) with at least one compound of formula (II) 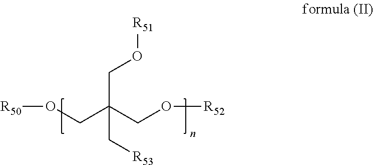
wherein n is an integer from 1 to 20,
wherein R₅₀, R₅₁, and R₅₂ are independently from each other selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, —(C═O)R₅₅, or —[CH₂CHR₅₆O]ₘ—R₅₇;
R₅₃ is selected from hydrogen, substituted or unsubstituted, linear or branched C₁-C₂₄ alkyl, substituted or unsubstituted, linear or branched C₂-C₂₄ alkenyl, or OR₅₉,
wherein R₅₉ is selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, —(C═O)R₅₅, or —[CH₂CHR₅₆O]ₘ—R₅₇,
R₅₄ is selected from hydrogen, substituted or unsubstituted, linear or branched C₁-C₂₄ alkyl, substituted or unsubstituted, linear or branched C₂-C₂₄ alkenyl, or —(C═O)—C₁-C₂₄ alkyl;
R₅₅ is selected from substituted or unsubstituted, linear or branched C₁-C₂₄ alkyl, or substituted or unsubstituted, linear or branched C₂-C₂₄ alkenyl;
R₅₆ is selected from hydrogen, or substituted or unsubstituted, linear or branched C₁-C₂₄ alkyl,
R₅₇ is selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, or —(C═O)R₅₅;
wherein t is an integer from 1 to 20,
wherein j is an integer from 1 to 20,
wherein m is an integer from 1 to 100,
with the proviso that at least one of R₅₀, R₅₁, R₅₂, R₅₄, R₅₇ and R₅₉ is hydrogen; and if n=1, at least one of R₅₀, R₅₁ and R₅₂ is not hydrogen.

20. The product, or a salt thereof according to claim 19,
wherein G* is 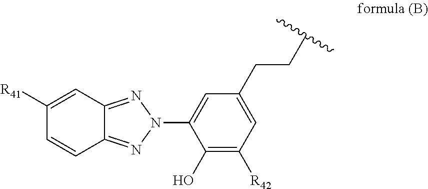
R₄₁, and R₄₂ independently of each other, are selected from hydrogen, halogen, substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, or substituted or unsubstituted C₇-C₂₄ arylalkyl.

21. The product, or a salt thereof according to claim 20, wherein R₄₁, and R₄₂ independently of each other, are selected from hydrogen, halogen, substituted or unsubstituted, linear or branched C₁-C₂₄ alkyl, or substituted or unsubstituted, linear or branched C₂-C₂₄ alkenyl.

22. The product, or a salt thereof according to claim 19, wherein at least one compound of formula (II) is 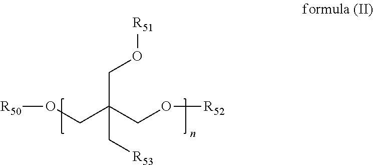
wherein n is an integer from 1 to 10;
wherein R₅₀, R₅₁, and R₅₂ are independently from each other selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, —(C═O)R₅₅, or —[CH₂CHR₅₆O]ₘ—R₅₇;
R₅₃ is selected from hydrogen, substituted or unsubstituted, linear or branched C₁-C₂₄ alkyl, substituted or unsubstituted, linear or branched C₂-C₂₄ alkenyl, or OR₅₉,
wherein R₅₉ is selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, —(C═O)R₅₅, or —[CH₂CHR₅₆O]ₘ—R₅₇,
R₅₄ is selected from hydrogen, substituted or unsubstituted, linear or branched C₁-C₂₄ alkyl, or —(C═O)—C₁₋₂₄ alkyl;
R₅₅ is substituted or unsubstituted, linear or branched C₁-C₂₄ alkyl;
R₅₆ is selected from H, or substituted or unsubstituted, linear or branched C₁-C₂₄ alkyl,
R₅₇ is selected from hydrogen, substituted or unsubstituted, linear or branched C₁-C₂₄ alkyl, substituted or unsubstituted, linear or branched C₂-C₂₄ alkenyl, —[(C═O)—(CH₂)—ₜ—NH]ⱼ—R₅₄, —[(C═O)—(CH₂)ₜ—O]ⱼ—R₅₄, or —(C═O)R₅₅;
wherein t is an integer from 1 to 10,
wherein j is an integer from 1 to 10,
wherein m is an integer from 1 to 50,
with the proviso that at least one of R₅₀, R₅₁, R₅₂, R₅₄ and R₅₇ is hydrogen; and if n=1, at least one of R₅₀, R₅₁ and R₅₂ is not hydrogen.

23. The product, or a salt thereof according to claim 22, wherein n is an integer from 2 to 5;
wherein R₅₀, R₅₁, and R₅₂ are independently from each other selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, —(C═O)R₅₅, or —[CH₂CHR₅₆O]ₘ—R₅₇;
R₅₃ is selected from hydrogen, substituted or unsubstituted, linear or branched C₁₋₆ alkyl, substituted or unsubstituted, linear or branched C₂-6 alkenyl, or OR₅₉,
wherein R₅₉ is selected from hydrogen, substituted or unsubstituted, linear or branched C₁-C₂₄ alkyl, substituted or unsubstituted, linear or branched C₂-C₂₄ alkenyl, —[(C═O)—(CH₂)ₜ—NH]ⱼ—R₅₄, —[(C═O)—(CH₂)ₜ—O]ⱼ—R₅₄, —(C═O)R₅₅, or —[CH₂CHR₅₆]ₘ—R₅₇,
R₅₄ is selected from hydrogen, or substituted or unsubstituted, linear or branched C₁-C₂₄ alkyl;
R₅₅ is substituted or unsubstituted, linear or branched C₁-C₂₄ alkyl;
R₅₆ is selected from H, or substituted or unsubstituted, linear or branched C₁₋₆ alkyl,
R₅₇ is selected from hydrogen, substituted or unsubstituted, linear or branched C₁-C₂₄, —[(C═O)—(CH₂)ₜ—O]ⱼ—R₅₄, or —(C═O)R₅₅;
wherein t is an integer from 1 to 5,
wherein j is an integer from 1 to 5,
wherein m is an integer from 1 to 20, with the proviso that at least one of R₅₀, R₅₁, R₅₂, R₅₄ and R₅₇ is hydrogen; and if n=1, at least one of R₅₀, R₅₁ and R₅₂ is not hydrogen.

24. The product, or a salt thereof according to claim 23, wherein n is an integer from 2 to 3;
wherein R₅₀, R₅₁, and R₅₂ are independently from each other selected from hydrogen, substituted or unsubstituted, linear or branched C₁-C₂₄ alkyl, or —[(C═O)—(CH₂)ₜ—O]ⱼ—R₅₄;
R₅₃ is selected from hydrogen, C₁ alkyl, or OR₅₉, wherein R₅₉ is as defined as above,
R₅₄ is selected from hydrogen, or substituted or unsubstituted, linear or branched C₁₋₆ alkyl;
R₅₅ is substituted or unsubstituted, linear or branched C₁₋₆ alkyl;
R₅₆ is selected from H, or C₁ alkyl,
R₅₇ is selected from hydrogen, substituted or unsubstituted, linear or branched C₁-C₂₄ alkyl, or —[(C═O)—(CH₂)ₜ—O]ⱼ—R₅₄,
wherein t is an integer from 3 to 5,
wherein j is an integer from 1 to 2,
wherein m is an integer from 4 to 16,
with the proviso that at least one of R₅₀, R₅₁, R₅₂, R₅₄ and R₅₇ is hydrogen; and if n=1, at least one of R₅₀, R₅₁ and R₅₂ is not hydrogen.

25. The product, or a salt thereof according to claim 19, wherein the compound of formula (II) has a hydroxy number calculated in the range of 50 to 5000 mg.

26. The product, or a salt thereof according to claim 19, wherein the compound of formula (II) has viscosity in the range of 1 to 5000 mPa·S measured at 23° C. according to EN ISO 3219.

27. The product, or a salt thereof according to claim 19, wherein the compound of formula (I) is selected from methyl 3-[3-(benzotriazol-2-yl)-5-tert-butyl-4-hydroxy-phenyl]propanoate, ethyl 3-[3-(benzotriazol-2-yl)-5-tert-butyl-4-hydroxy-phenyl]propanoate, 3-[3-(benzotriazol-2-yl)-5-tert-butyl-4-hydroxy-phenyl]propanoic acid, 3-[3-tert-butyl-5-(5-chlorobenzotriazol-2-yl)-4-hydroxy-phenyl]propanoic acid, methyl 3-[3-tert-butyl-5-(5-chlorobenzotriazol-2-yl)-4-hydroxy-phenyl]propanoate, ethyl 3-[3-tert-butyl-5-(5-chlorobenzotriazol-2-yl)-4-hydroxy-phenyl]propanoate, methyl 2-[4-[4,6-bis(2,4-dimethylphenyl)-1,3,5-triazin-2-yl]-3-hydroxy-phenoxy]propanoate, methyl 2-[4-[4,6-bis(4-phenylphenyl)-1,3,5-triazin-2-yl]-3-hydroxy-phenoxy]propanoate, methyl 2-[4-(4,6-diphenyl-1,3,5-triazin-2-yl)-3-hydroxy-phenoxy]propanoate, ethyl 2-[4-[4,6-bis(2,4-dimethylphenyl)-1,3,5-triazin-2-yl]-3-hydroxy-phenoxy]-3-butoxy-propanoate, ethyl 2-[4-(4,6-diphenyl-1,3,5-triazin-2-yl)-3-hydroxy-phenoxy]octanoate, methyl 2-[4-(4,6-diphenyl-1,3,5-triazin-2-yl)-3-hydroxy-phenoxy]butanoate, methyl 2-[4-[4,6-bis(2,4-dimethylphenyl)-1,3,5-triazin-2-yl]-3-hydroxy-phenoxy]-2-methyl-propanoate, methyl 2-[4-[4,6-bis(2,4-dimethylphenyl)-1,3,5-triazin-2-yl]-3-hydroxy-phenoxy]octanoate, methyl 2-[4-[4,6-bis(2,4-dimethylphenyl)-1,3,5-triazin-2-yl]-3-hydroxy-phenoxy]hexanoate, methyl 2-[4-[4,6-bis(2,4-dihydroxyphenyl)-1,3,5-triazin-2-yl]-3-hydroxy-phenoxy]propanoate, octyl 2-[4-[4,6-bis[2-hydroxy-4-(1-methyl-2-octoxy-2-oxo-ethoxy)phenyl]-1,3,5-triazin-2-yl]-3-hydroxy-phenoxy]propanoate (tinuvin 477), 6-methylheptyl 2-[4-[4,6-bis(4-phenylphenyl)-1,3,5-triazin-2-yl]-3-hydroxy-phenoxy]propanoate (tinuvin 479), octyl 2-[4-[4,6-bis(4-phenylphenyl)-1,3,5-triazin-2-yl]-3-hydroxy-phenoxy]propanoate, methyl 2-[2-[4-[4,6-bis(2,4-dimethylphenyl)-1,3,5-triazin-2-yl]-3-hydroxy-phenoxy]ethoxy]propanoate, methyl 2-[4-[4,6-bis(4-phenylphenyl)-1,3,5-triazin-2-yl]-3-hydroxy-phenoxy]-3-butoxy-propanoate, methyl 2-[3-hydroxy-4-[4-[2-hydroxy-4-(1-methoxycarbonylpropoxy)phenyl]-6-(4-methoxyphenyl)-1,3,5-triazin-2-yl]phenoxy]butanoate, methyl 2-[4-[4,6-bis(4-butoxy-2-hydroxy-phenyl)-1,3,5-triazin-2-yl]-3-hydroxy-phenoxy]propanoate, methyl 2-[4-[4,6-bis(4-hexoxy-2-hydroxy-3-methyl-phenyl)-1,3,5-triazin-2-yl]-3-hydroxy-2-methyl-phenoxy]propanoate, ethyl 2-[4-[4,6-bis(4-phenylphenyl)-1,3,5-triazin-2-yl]-3-hydroxy-phenoxy]butanoate, methyl 2-[4-[4,6-bis(4-phenylphenyl)-1,3,5-triazin-2-yl]-3-hydroxy-phenoxy]octanoate, methyl 2-[4-[4,6-bis[2-hydroxy-4-(1-methoxycarbonylpropoxy)phenyl]-1,3,5-triazin-2-yl]-3-hydroxy-phenoxy]butanoate, methyl 2-[4-[4,6-bis(2,4-dimethylphenyl)-1,3,5-triazin-2-yl]-3-hydroxy-phenoxy]butanoate, ethyl 2-[4-[4,6-bis(4-phenylphenyl)-1,3,5-triazin-2-yl]-3-hydroxy-phenoxy]propanoate, methyl 2-[4-[4,6-bis[2-hydroxy-4-(2-methoxy-1-methyl-2-oxo-ethoxy)phenyl]-1,3,5-triazin-2-yl]-3-hydroxy-phenoxy]propanoate, methyl 2-[3-hydroxy-4-[4-[2-hydroxy-4-(2-methoxy-1-methyl-2-oxo-ethoxy)phenyl]-6-(4-methoxyphenyl)-1,3,5-triazin-2-yl]phenoxy]propanoate, or a combination of two or more thereof.

28. The product, or a salt thereof according to claim 27, wherein the compound of formula (I) is selected from methyl 3-[3-(benzotriazol-2-yl)-5-tert-butyl-4-hydroxy-phenyl]propanoate, ethyl 3-[3-(benzotriazol-2-yl)-5-tert-butyl-4-hydroxy-phenyl]propanoate, 3-[3-(benzotriazol-2-yl)-5-tert-butyl-4-hydroxy-phenyl]propanoic acid, 3-[3-tert-butyl-5-(5-chlorobenzotriazol-2-yl)-4-hydroxy-phenyl]propanoic acid, methyl 3-[3-tert-butyl-5-(5-chlorobenzotriazol-2-yl)-4-hydroxy-phenyl]propanoate, ethyl 3-[3-tert-butyl-5-(5-chlorobenzotriazol-2-yl)-4-hydroxy-phenyl]propanoate, or a combination of two or more thereof.

29. The product, or a salt thereof according to claim 19, wherein the mol ratio of compound of formula (I) to compound of formula (II) is in the range of 1 to 10 to 10 to 1.

30. The product, or a salt thereof according to claim 29, wherein the mol ratio of compound of formula (I) to compound of formula (II) is in the range of 1 to 5 to 5 to 1.

31. The product, or a salt thereof according to claim 19, wherein the compound of formula (II) is selected from alkoxylated trimethylolethane, ethoxylated trimethylolethane, propoxylated trimethylolethane, alkoxylated trimethylolpropane, ethoxylated trimethylolpropane, propoxylated trimethylolpropane, alkoxylated 1,1,1-Trimethylolisobutane, ethoxylated 1,1,1-Trimethylolisobutane, propoxylated 1,1,1-Trimethylolisobutane, alkoxylated 1,1,1-Trimethylolpentane, alkoxylated 1,1,1-Tri(hydroxymethyl)-2,2-dimethylpropane, alkoxylated 2-(Hydroxymethyl)-2-(2-methylpropyl)-1,3-propanediol, alkoxylated 2-(Hydroxymethyl)-2-(methoxymethyl)-1,3-propanediol, alkoxylated trimethylolheptane, alkoxylated 2-allyl-2-(hydroxymethyl)propane-1,3-diol, alkoxylated tris(hydroxymethyl)undecane, alkoxylated trimethylolacetic acid, alkoxylated pentaerythritol monoallyl ether, alkoxylated α,α,α-tris(hydroxymethyl)toluene, alkoxylated pentaerythritol, ethoxylated pentaerythritol, propoxylated pentaerythritol, alkoxylated dipentaerythritol, ethoxylated dipentaerythritol, propoxylated dipentaerythritol, alkoxylated tripentaerythritol, ethoxylated tripentaerythritol, propoxylated tripentaerythritol, [3-hydroxy-2,2-bis(hydroxymethyl)propyl]dodecanoate, 3-hydroxy-2,2-bis(hydroxymethyl)propyl octadecenoate, pentaerythritol monoleate, 2-(methoxymethyl)-2-methyl-1,3-propanediol, 2-(2,5-Dioxahexyl)-2-methyl-1,3-propanediol, ditrimethylolpropane, alkoxylated ditrimethylolethane, ethoxylated ditrimethylolethane, pentaerythritol tris(2-hydroxyethyl ether, Diallyl pentaerythritol, trimethylolpropane diallyl ether, tristrimethylolpropane, alkoxylated tristrimethylolpropane, ethoxylated tristrimethylolpropane, propoxylated tristrimethylolpropane, 2-methyl-, 1,1′-[2,2-bis(hydroxymethyl)-1,3-propanediyl]2-propenoic ester.

32. The product, or a salt thereof according to claim 19 has weight average molecular weight in the range of 500 to 20′000.

33. The product, or a salt thereof according to claim 19 has solubility is greater than 10 g per 100 mL xylene at room temperature.

34. Use of the product or a salt thereof according to claim 19 as ultraviolet stabilizer.

35. A composition comprising a product or a salt thereof according to claim 19.

36. A method of protecting a material or coating from light, wherein the method comprises a step of providing the product according to claim 19 or salt thereof as UV stabilizer.
